(19) United States
(12) Patent Application Publication
(10) Pub. No.: US 2002/0164617 A1
(43) Pub. Date: Nov. 7, 2002

(54) Affinity selection-based screening of hydrophobic proteins

(75) Inventors: Jason S. Felsch, Waltham, MA (US); David Allen Annis JR., Cambridge, MA (US); Krishna Kalghatgi, Westboro, MA (US); Huw M. Nash, Cambridge, MA (US)

Correspondence Address: HALE AND DORR, LLP, 60 STATE STREET, BOSTON, MA, 02109

(73) Assignee: NeoGenesis Pharmaceuticals, Inc., Cambridge, MA (US)

(21) Appl. No.: 10/029,009

(22) Filed: Dec. 19, 2001

Related U.S. Application Data
(60) Provisional application No. 60/258,970, filed on Dec. 29, 2000.

(51) Int. Cl. C12Q 1/68

(52) U.S. Cl. 435/6

(57) ABSTRACT

The invention relates to methods based on affinity selection for the identification of ligands for hydrophobic proteins bound by amphiphile. The invention also provides hydrophobic proteins and methods of isolation of hydrophobic proteins that are suitable for ligand screening.

### CROSS-REFERENCE TO RELATED APPLICATIONS

This application claims the benefit of U.S. Provisional Application No. 60/258,970 filed Dec.29, 2000.

### BACKGROUND OF THE INVENTION

1. Field of the Invention

The invention relates to the fields of pharmacology and medicine. More specifically, the invention relates to the screening of hydrophobic proteins for the identification of the respective ligand molecules with particular relevance to the development of novel medicines and medical diagnostics.

2. Summary of the Related Art

Hydrophobic proteins (HPs) present a unique problem for the pharmaceutical industry in the development of agonists and antagonists of hydrophobic protein function. The difficulty arises from the fact that HPs are not easily purified and are difficult to work with in isolated form (e.g., solubility difficulties, etc.). Given the nonpolar nature of hydrophobic proteins, they may be found, inter alia, associated with the lipid bi-layer of a cell and the organelles therein. By way of nonlimiting example, the term hydrophobic protein includes membrane proteins, integral membrane proteins, transmembrane proteins, monotopic membrane proteins, polytopic membrane proteins, pump proteins(a subclass of enzymes), channel proteins, receptor kinase proteins, G protein-coupled receptor proteins, membrane-associated enzymes, transporter proteins, etc. Frequently, these proteins play an important role in intra- and intercellular signaling and the general relation of a cell to its environment, e.g., solute movement, etc. Thus, hydrophobic proteins are important targets for drug development.

The human genome project will provide an enormous amount of information about the structure and function of hydrophilic and hydrophobic proteins encoded therein. For example, it is estimated that 1,700-5,000 G protein-coupled receptor proteins (GPCRs) will be discovered in the human genome (Marchese, A., et al. (1999) Trends Pharmacol. Sci. 20:370; Henikoff, S., et al. (1997) Science 278:609). However, given the lack of suitable screening methodologies for the identification of ligands that bind hydrophobic proteins, hundreds of the GPCRs identified by the human genome project will be classified as orphan receptors, having no known ligand to advance their study. GPCRs are so important to the medical sciences that a separate database has been established to provide information on sequence data, mutant data, and ligand binding data, for example (Horn, F. et al. (1998) Nucleic Acids Research 26: 227-281). Thus, there is a need in the art for the development of screening methodologies, particularly high throughput methodologies, for HP ligand identification.

In the prior art, there is no record of affinity selection-based screening of HPs. Instead, these targets are screened in functional assays or ligand displacement assays. All ligand displacement assays and most functional assays used to screen HPs are either performed in cell-based formats (for example, see Jayawickreme, C. K. and Kost, T. A., (1997) Current Opinion in Biotechnology 8: 629-634 and Chen, G., et al. (1999) Molecular Pharmacology 57: 125-124, which both disclose cell-based melanophore assays; Mere, L. et al. (1999) Drug Discovery Today 4:363-369, which discloses a cell-based fluorescence resonance engery transfer (FRET)-based assay; and Schaeffer, M. T., et al. (1999) J. Receptor & Signal Transduction Research 19: 927-938, which discloses a cell-based aequorin assay) or use impure cell membrane preparations (for example, see Cromlish et al. U.S. Pat. No. 5,543,297, which discloses a microsome-based assay; and see Labella, F. S., et al. Fed. Proc. (1985) 44: 2806-2811, which discloses a radioligand displacement assay using membrane preparations.). These screening formats are poorly defined at the molecular level and suffer from low signal-to-noise ratios, false positives, and variability in the degree to which the target protein is expressed and wide variability in gene expression parameters.

More rarely, HP targets are purified for screening. For example, COX-2 purified in a detergent-solubilized form can be screened by monitoring its enzymatic activity in a homogeneous solution phase assay wherein small molecule inhibitors of enzymatic activity can be identified as drug leads (see Song, Y., et al. (1999) J. Med. Chem. 42: 1151-1160 and Barnett, J., et al. (1994) Biochimica et Biophysica Acta 1209: 130-139. Alternatively, the HP may be bound to a carrier for screening purposes (see Sklar, L. A. et al. (2000) Biotechniques 28: 976-985; Bieri, C. et al. (1999) Nature Biotechnology 17: 1105-1108; and Schmid, E. L., et al. (1998) Anal. Chem. 70: 1331-1338). However, screening assays that use functional readouts presume foreknowledge of the target's function. Also, as in the case of many imaging agents used for medical diagnosis, many desired protein ligands do not modulate an assayable function and only bind to the protein.

Affinity selection to identify ligands to water-soluble proteins is known in the art. For example, International Publication No. WO 99/35109 by Nash et al. describes a method for producing mass-coded combinatorial libraries, which are useful in combination with affinity selection and the identification of the bound ligand by mass spectroscopy. International Patent Application No. WO 97/01755 by Jindal et al. describes the affinity selection of ligands bound to a target molecule combined with the subsequent isolation of the ligand molecule by multidimensional chromatographic methodology. And U.S. Pat. No. 6,020,141 by Pantoliano et al. describes a method of affinity selection combined with ligand identification by thermal shift assay.

Regardless of these advances with affinity ligand selection and ligand identification, there still remains a fundamental challenge to apply affinity selection to non-water-soluble HP targets because of the hindering presence of excess amphiphile, which is required to maintain the pure HP in a biologically active conformation.

It is important to recognize the difference between a preparation of a water-soluble protein and a preparation of pure HP. The HP is solvated through hydrophobic interactions between the hydrophobic parts of the HP and the hydrophobic moiety of the amphiphile. In a preparation of pure water-soluble protein, all buffer components are hydrophilic and solvate the protein either through hydration or by participating in electrostatic or ionic bonds. By contrast the amphiphile in preparations of pure HP imparts a colloidal characteristic to the solution. Typically, HPs are purified in 100 to 10,000-fold molar excess of detergent. These amphiphilic detergent molecules interact with both the HP and the drug molecules being screened. In addition, amphiphiles form macromolecular assemblies, like micelles or liposomes, that are just as large as most proteins. These macromolecular assemblies impart a colloidal characteristic to solutions of amphiphile-solubilized HP, further distinguishing HP's from soluble proteins.

Compared to soluble protein targets, the extra complexity of HP-amphiphile preparations hampers the detection of the bound ligands, lowers screening sensitivity, and yields high rate of false positive. In a typical preparation, the molecular entities responsible for these complications could be identified as: HP-amphiphile complexes (20 μM, HP:amphiphile::1:5-250; MW=50-500 kD), micelles (5000 μM MW=60 kD), monomeric amphiphile (500 μM; MW=1200). In an analogous water-soluble protein preparation, one would have only 20 μM protein. In both cases buffers (e.g. tris or Na-phosphate) and salt (e.g. NaCl or KCl) would also be present. For preparations of HP proteins, the presence of the various amphiphile entities presents extra complexity not found in soluble protein preparations.

Thus, there is a continuing need in the art for an affinity selection-based HP screening method that can operate in the presence of an amphiphile without regard to the specific biological function of the HP target.

### BRIEF SUMMARY OF THE INVENTION

The invention provides an affinity selection-based HP screening method that can operate in the presence of an amphiphile without regard to the specific biological function of the HP target.

The present invention solves problems associated with affinity selection of HP ligands by enabling detection of the specific binding of a small drug-like molecules to an HP in the presence of an amphiphile. In addition, the present invention also provides novel methods and compositions of matter for the production of purified HPs useful for screening purposes. These discoveries have been exploited to provide the present invention, which includes compositions and methods.

In a first aspect, the invention provides a method for identifying a ligand for a hydrophobic protein, the method comprising (a) selecting a ligand molecule by affinity selection by exposing a hydrophobic target protein bound by an amphiphile to a multiplicity of molecules to promote the formation of at least one complex between the hydrophobic target protein and the ligand molecule; (b) separating the complex from the unbound molecules; and (c) identifying the ligand molecule.

In certain embodiments of the first aspect, exposure of the hydrophobic target protein to a multiplicity of molecules occurs under homogeneous solution phase conditions. In certain embodiments of the first aspect, exposure of the hydrophobic target protein to a multiplicity of molecules occurs under heterogeneous solution phase conditions. In certain embodiments of the first aspect, selection of the ligand molecule is done using multi-dimensional chromatography.

In certain embodiments of the first aspect, the hydrophobic target protein is selected from the group consisting of a membrane protein, an integral membrane protein, a transmembrane protein, a monotopic membrane protein, a polytopic membrane protein, a pump protein, a channel protein, a receptor kinase protein, a G protein-coupled receptor protein, a membrane-associated enzyme, and a transporter protein.

In certain embodiments of the first aspect, the multiplicity of molecules is a mass coded library of molecules. In certain embodiments of the first aspect, the multiplicity of molecules is a library of molecules that is not mass coded. In certain embodiments of the first aspect, the amphiphile is selected from the group consisting of (a) a polar lipid, (b) an amphiphilic macromolecular polymer, (c) a surfactant or detergent, and (d) an amphiphilic polypeptide. In certain embodiments of the first aspect, ligand molecule identification is done by mass spectral analysis. In certain embodiments of the first aspect, the ligand molecule is deconvoluted by mass spectral analysis. In certain embodiments of the first aspect, separation of the complex from the unbound molecules is accomplished with solid phase chromatography media.

In certain embodiments of the first aspect, the hydrophobic target protein comprises (a) at least one transmembrane domain sequence, (b) at least two tag sequences useful for affinity purification, and (c) a hydrophobic protein (HP) sequence. In certain embodiments thereof, the hydrophobic protein sequence is selected from the group consisting of (a) a membrane protein, (b) an integral membrane protein, (c) a transmembrane protein, (d) a monotopic membrane protein, (e) a polytopic membrane protein, (f) a pump protein, (g) a channel protein, (h) a receptor kinase protein, (i) a G protein-coupled receptor protein, (j) a membrane-associated enzyme, and (k) a transporter protein. In certain embodiments thereof, the tag sequences comprise epitope tag sequences selected from the group consisting of (a) a FLAG tag (NH2-DYKDDDDK-COOH) (SEQ ID NO:1), (b) an EE tag (NH2-EEEEYMPME-COOH) (SEQ ID NO:2), (c) a hemagglutinin tag (NH2-YPYDVPDYA-COOH) (SEQ ID NO:3), (d) a myc tag (NH2-KHKLEQLRNSGA-COOH) (SEQ ID NO:4), (e) an HSV tag (NH2-QPELAPEDPED-COOH) (SEQ ID NO:5) and (f) a rhodopsin tag (NH2 MNGTEGPNFYVPFSNKTGVVRSPFEAPQYYLAEPWQFSM-COOH) (SEQ ID NO:6).

In certain embodiments of the first aspect, the hydrophobic target protein comprises a sequence with an amino terminus to carboxy terminus order selected from the group consisting of (a) Tag1-Tag2-HP, (b) Tag1-HP-Tag2, and (c) HP-Tag1-Tag2. In certain embodiments of thereof, the invention provides a method wherein the hydrophobic target protein is selected from the group consisting of (a) Myc tag-EE tag-Human m2 muscarinic acetylcholine receptor (mAChR) (SEQ ID NO:7), (b) Flag tag-Human Beta 2 Adrenergic Receptor-EE tag (SEQ ID NO:8), (c) Human Neurokinin 3 Receptor-HSV tag-Myc tag (SEQ ID NO:9), (d) Flag tag-Human m1 mAChR-EE tag (SEQ ID NO:10), and (e) Rat m3 mAChR-HSV tag-OctaHis tag (SEQ ID NO:11). In certain embodiments thereof, the invention provides a method wherein the hydrophobic target protein further comprises a heterologous signal sequence (SS) at the amino terminus. In certain embodiments thereof, the heterologous signal sequence is selected from the group consisting of (a) the Mellitin signal sequence of NH2-KFLVNVALVFMVVYISYIYA-COOH (SEQ ID NO:12), (b) the GP signal sequence of NH2-VRTAVLILLLVRFSEP-COOH (SEQ ID NO:13), (c) the Hemagglutinin signal sequence of NH2-KTIIALSYIFCLVFA-COOH (SEQ ID NO:14), (d) the rhodopsin tag 1 signal sequence of NH2-MNGTEGPNFYVPFSNKTGVVRSPFEAPQYYLAEP-COOH (SEQ ID NO:15), and (e) the rhodopsin tag ID4 signal sequence of NH2-GKNPLGVRKTETSQVAPA-COOH (SEQ ID NO:16). In certain embodiments thereof the tag sequences further comprise a hexahistidine sequence (SEQ ID NO:17) and a decahistidine sequence (SEQ ID NO:18). In yet certain embodiments thereof the hydrophobic target protein is selected from the group consisting of (a) GP67 SS-Myc tag-EE tag-Human m2 mAChR (SEQ ID NO:19), (b) Mellitin SS-Flag tag-Human Beta 2 Adrenergic Receptor-EE tag(SEQ ID NO:20), (c) Hemagglutinin SS-Human Neurokinin 3 Receptor-HSV tag-Myc tag (SEQ ID NO:21), (d) Mellitin SS-Flag tag-Human m1 mAChR-EE tag (SEQ ID NO:22), and (e) Hemagglutinin SS-Rat m3 mAChR-HSV tag-OctaHis tag (SEQ ID NO:23).

In a second aspect, the invention provides a method of isolating a hydrophobic protein, the method comprising (a) purifying the hydrophobic protein by sucrose gradient ultracentrifugation, (b) purifying the hydrophobic protein by antibody affinity purification, and (c) purifying the hydrophobic protein by immobilized metal affinity chromatography.

In certain embodiments of the second aspect, the hydrophobic protein comprises (a) at least one transmembrane domain sequence, (b) at least two tag sequences useful for affinity selection, and (c) a hydrophobic protein (HP) sequence. In certain embodiments thereof, the hydrophobic protein sequence is selected from the group consisting of (a) a membrane protein, (b) an integral membrane protein, (c) a transmembrane protein, (d) a monotopic membrane protein, (e) a polytopic membrane protein, (f) a pump protein, (g) a channel protein, (h) a receptor kinase protein, (i) a G protein-coupled receptor protein, (j) a membrane-associated enzyme, and (k) a transporter protein. In certain embodiments of the second aspect, the tag sequences of the hydrophobic protein comprise epitope tag sequences selected from the group consisting of (a) a FLAG tag (NH2-DYKDDDDK-COOH) (SEQ ID NO:l), (b) an EE tag (NH2-EEEEYMPME-COOH) (SEQ ID NO:2), (c) a hemagglutinin tag (NH2-YPYDVPDYA-COOH) (SEQ ID NO:3), (d) a myc tag (NH2-KHKLEQLRNSGA-COOH) (SEQ ID NO:4), (e) an HSV tag (NH2-QPELAPEDPED-COOH) (SEQ ID NO:5) and (f) a rhodopsin tag (NH2 MNGTEGPNFYVPFSNKTGVVRSPFEAPQYYLAEPWQFSM-COOH) (SEQ ID NO:6). In certain embodiments of the second aspect, the hydrophobic protein comprises a sequence with an amino terminus to carboxy terminus order selected from the group consisting of (a) Tag1-Tag2-HP, (b) Tag1-HP-Tag2, and (c) HP-Tag1-Tag2.

In certain embodiments of the second aspect, the hydrophobic protein is selected from the group consisting of (a) Myc tag-EE tag-Human m2 mAChR (SEQ ID NO:7), (b) Flag tag-Human Beta 2 Adrenergic Receptor-EE tag (SEQ ID NO:8), (c) Human Neurokinin 3 Receptor-HSV tag-Myc tag (SEQ ID NO:9), (d) Flag tag-Human m1 mAChR-EE tag (SEQ ID NO:10), and (e) Rat m3 mAChR-HSV tag-OctaHis tag (SEQ ID NO:11). In embodiments thereof, the hydrophobic protein further comprises a heterologous signal sequence (SS) at the amino terminus. In certain embodiments thereof, the heterologous signal sequence is selected from the group consisting of (a) the Mellitin signal sequence of NH2-KFLVNVALVFMVVYISYIYA-COOH (SEQ ID NO:12), (b) the GP signal sequence of NH2-VRTAVLILLLVRFSEP-COOH (SEQ ID NO:13), (c) the Hemagglutinin signal sequence of NH2-KTIIALSYIFCLVFA-COOH (SEQ ID NO:14), (d) the rhodopsin tag 1 signal sequence of NH2-MNGTEGPNFYVPFSNKTGVVRSPFEAPQYYLAEP-COOH (SEQ ID NO:15), and (e) the rhodopsin tag ID4 signal sequence of NH2-GKNPLGVRKTETSQVAPA-COOH (SEQ ID NO:16). In certain embodiments of the second aspect, the tag sequences of the hydrophobic protein further comprise a hexahistidine sequence (SEQ ID NO:17) and a decahistidine sequence (SEQ ID NO:18).

In certain embodiments thereof, the hydrophobic target protein is selected from the group consisting of (a) GP67 SS-Myc tag-EE tag-Human m2 mAChR (SEQ ID NO:19), (b) Mellitin SS-Flag tag-Human Beta 2 Adrenergic Receptor-EE tag(SEQ ID NO:20), (c) Hemagglutinin SS-Human Neurokinin 3 Receptor-HSV tag-Myc tag (SEQ ID NO:21), (d) Mellitin SS-Flag tag-Human m1 mAChR-EE tag (SEQ ID NO:22), and (e) Hemagglutinin SS-Rat m3 mAChR-HSV tag-OctaHis tag (SEQ ID NO:23).

In a third aspect, the invention provides an isolated nucleic acid molecule suitable for hydrophobic protein expression, comprising (a) a vector polynucleotide sequence for protein expression in a eukaryotic cell, and (b) a polynucleotide sequence encoding an engineered hydrophobic protein comprising the following elements (i) an N-terminal methionine residue, (ii) a heterologous signal sequence (SS), (iii) at least one transmembrane domain sequence, (iv) at least two tag sequences useful for affinity purification, and (v) a hydrophobic protein (HP) sequence. In certain embodiments thereof, the N-terminal methionine sequence and the heterologous signal sequence are selected from the group consisting of (a) MKFLVNVALVFMVVYISYIYA (SEQ ID NO:24), (b) MVRTAVLILLLVRFSEP (SEQ ID NO:25), (c) MKTIIALSYIFCLVFA (SEQ ID NO:26), (d) MMNGTEGPNFYVPFSNKTGVVRSPFEAPQYYLAEP-COOH (SEQ ID NO:27), and (e) MGKNPLGVRKTETSQVAPA-COOH (SEQ ID NO:28).

In certain embodiments thereof, the tag sequences comprise epitope tag sequences selected from the group consisting of (a) a FLAG tag (NH2-DYKDDDDK-COOH) (SEQ ID NO:1), (b) an EE tag (NH2-EEEEYMPME-COOH) (SEQ ID NO:2), (c) a hemagglutinin tag (NH2-YPYDVPDYA-COOH) (SEQ ID NO:3), (d) a myc tag (NH2-KHKLEQLRNSGA-COOH) (SEQ ID NO:4), and (e) an HSV tag (NH2-QPELAPEDPED-COOH) (SEQ ID NO:5).

In certain embodiments of the third aspect, the elements of the engineered hydrophobic protein are arrayed from an amino to carboxy terminus order selected from the group consisting of (a) SS-Tag1-Tag2-HP, (b) SS-Tag1-HP-Tag2, and (c) SS-HP-Tag1-Tag2. In certain embodiments thereof, the engineered hydrophobic protein is selected from the group consisting of (a) GP67 SS-Myc tag-EE tag-Human m2 mAChR (SEQ ID NO:19), (b) Mellitin SS-Flag tag-Human Beta 2 Adrenergic Receptor-EE tag (SEQ ID NO:20), and (c) Hemagglutinin SS-Human Neurokinin 3 Receptor-HSV tag-Myc tag (SEQ ID NO:21). In a further embodiment of the third aspect, the tag sequences further comprise a hexahistidine sequence (SEQ ID NO:17) and a decahistidine sequence (SEQ ID NO:18).

In a fourth aspect, the invention provides a method for identifying a ligand for a hydrophobic protein, the method comprising (a) selecting a hydrophobic target protein from the group consisting of (i) a membrane protein, (ii) an integral membrane protein, (iii) a transmembrane protein, (iv) a monotopic membrane protein, (v) a polytopic membrane protein, (vii) a pump protein, (viii) a channel protein, (iX)a receptor kinase protein, (X) a G protein-coupled receptor protein, Xii) a membrane-associated enzyme, and (Xiii) a transporter protein, wherein the hydrophobic protein is bound by amphiphile selected from the group consisting of (i) a polar lipid, (ii) an amphiphilic macromolecular polymer, (iii) a surfactant or detergent, and (iV) an amphiphilic polypeptide; (b) selecting a ligand molecule using multi-dimensional chromatography by affinity selection by exposing under homogenous or heterogeneous solution phase conditions the hydrophobic target protein bound by an amphiphile to a multiplicity of molecules from a mass-coded library to promote the formation of at least one complex between the hydrophobic target protein and the ligand molecule, (c) separating the complex from the unbound molecules, and (d) identifying the ligand molecule by mass spectral analysis.

In a fifth aspect, the invention provides a method for identifying a ligand for a hydrophobic protein, the method comprising (a) selecting a hydrophobic target protein from the group consisting of (i) a membrane protein, (ii) an integral membrane protein, (iii) a transmembrane protein, (iv) a monotopic membrane protein, (v) a polytopic membrane protein, (vii) a pump protein, (viii) a channel protein, (iX) a receptor kinase protein, (X) a G protein-coupled receptor protein, Xii) a membrane-associated enzyme, and (Xiii) a transporter protein, wherein the hydrophobic protein is bound by amphiphile selected from the group consisting of (i) a polar lipid, (ii) an amphiphilic macromolecular polymer, (iii) a surfactant or detergent, and (iV) an amphiphilic polypeptide; (b) selecting a ligand molecule using multi-dimensional chromatography by affinity selection by exposing under homogenous or heterogeneous solution phase conditions the hydrophobic target protein bound by an amphiphile to a multiplicity of molecules from a library that is not mass-coded to promote the formation of at least one complex between the hydrophobic target protein and the ligand molecule, (c) separating the complex from the unbound molecules, and (d) identifying the ligand molecule by mass spectral analysis.

In a sixth aspect, the invention provides a method of isolating a hydrophobic protein, the method comprising: (a) selecting a hydrophobic protein comprising: (i) at least one transmembrane domain sequence, (ii) at least two tag sequences useful for affinity selection selected from the group consisting of: (A) a FLAG tag (NH2-DYKDDDDK-COOH) (SEQ ID NO:29), (B) an EE tag (NH2-EEEEYMPME-COOH) (SEQ ID NO:30), (C) a hemagglutinin tag (NH2-YPYDVPDYA-COOH) (SEQ ID NO:31), (D) a myc tag (NH2-KHKLEQLRNSGA-COOH) (SEQ ID NO:32), and (E) an HSV tag (NH2-QPELAPEDPED-COOH) (SEQ ID NO:33); (iii) a hydrophobic protein (HP) sequence selected from the group consisting of: (A) a membrane protein, (B) an integral membrane protein, (C) a transmembrane protein, (D) a monotopic membrane protein, (E) a polytopic membrane protein, (F) a pump protein, (G) a channel protein, (H) a receptor kinase protein, (I)a G protein-coupled receptor protein, (J) a membrane-associated enzyme, and (K) a transporter protein; (b) purifying the hydrophobic protein by sucrose gradient ultracentrifugation; (c) purifying the hydrophobic protein by antibody affinity purification; and (d) purifying the hydrophobic protein by immobilized metal affinity chromatography.

In a seventh aspect, the invention provides, an isolated nucleic acid molecule suitable for hydrophobic protein expression, comprising: (a) a vector polynucleotide sequence for protein expression in a eukaryotic cell, and (b) a polynucleotide sequence encoding an engineered hydrophobic protein comprising the following elements (i) an N-terminal methionine residue, (ii) a heterologous signal sequence (SS), wherein the N-terminal methionine sequence and the heterologous signal sequence are selected from the group consisting of (1) MKFLVNVALVFMVVYISYIYA (SEQ ID NO:24), (2) MVRTAVLILLLVRFSEP (SEQ ID NO:25), (3) MKTIIALSYIFCLVFA (SEQ ID NO:26), (4) MMNGTEGPNFYVPFSNKTGVVRSPFEAPQYYLAEP-COOH (SEQ ID NO:27) and (5) MGKNPLGVRKTETSQVAPA-COOH (SEQ ID NO:28); (iii) at least one transmembrane domain sequence, (iv) at least two tag sequences useful for affinity selection selected from the group consisting of (1) a FLAG tag (NH2-DYKDDDDK-COOH) (SEQ ID NO:1), (2) an EE tag (NH2-EEEEYMPME-COOH) (SEQ ID NO:2), (3) a hemagglutinin tag (NH2-YPYDVPDYA-COOH) (SEQ ID NO:3), (4) a myc tag (NH2-KHKLEQLRNSGA-COOH) (SEQ ID NO:4), and (5) an HSV tag (NH2-QPELAPEDPED-COOH) (SEQ ID NO:5)., and (v) a hydrophobic protein (HP) sequence selected from the group consisting of (1) a membrane protein, (2) an integral membrane protein, (3) a transmembrane protein, (4) a monotopic membrane protein, (5) a polytopic membrane protein, (6) a pump protein, (7) a channel protein, (8) a receptor kinase protein, (9) a G protein-coupled receptor protein, (10) a membrane-associated enzyme, and (11) a transporter protein.

### BRIEF DESCRIPTION OF THE DRAWINGS

FIG. 1 presents the amino acid sequence of the HP protein GP67 SS-Myc tag-EE tag-Human m2 mAChR (SEQ ID NO:19).

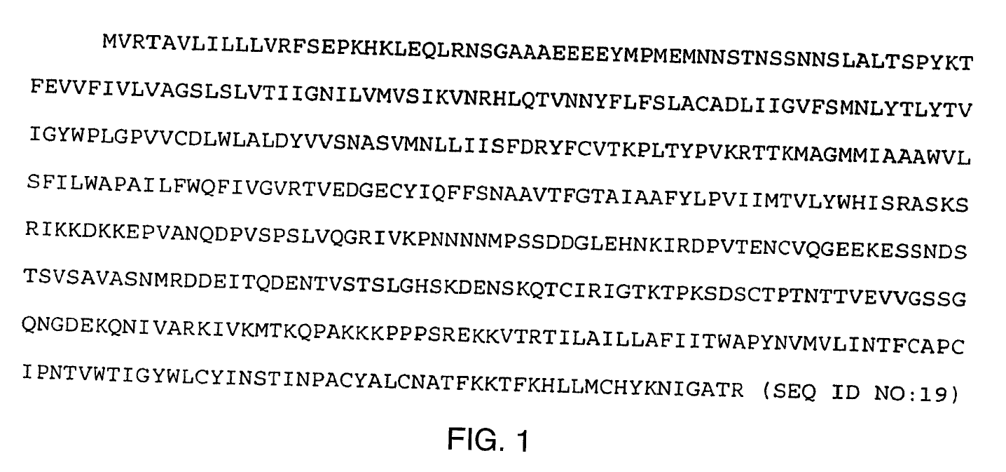

FIG. 2 presents the amino acid sequence of the HP protein Mellitin-Flag Tag-Human Beta 2 Adrenergic Receptor-EE (SEQ ID NO:20).

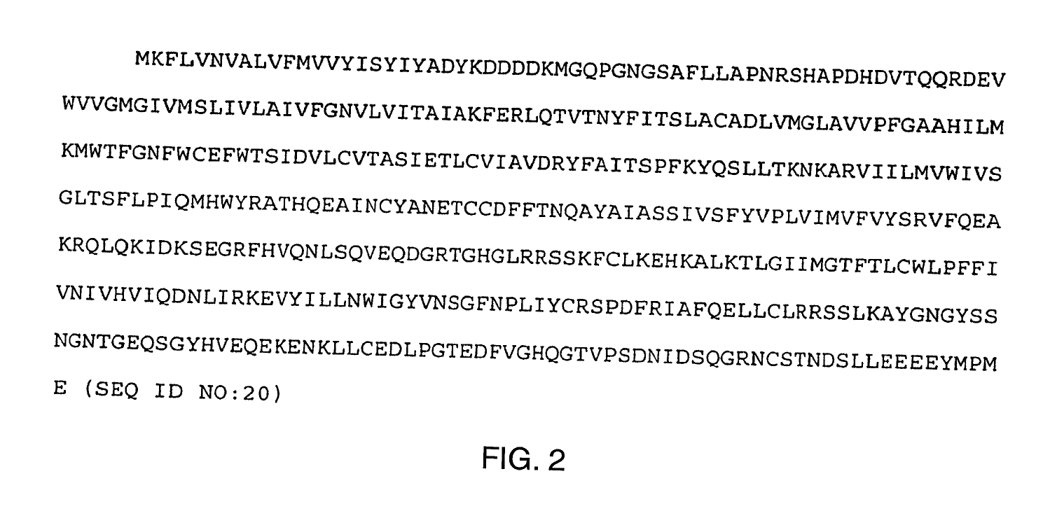

FIG. 3 presents the amino acid sequence of the HP protein Hemagglutinin SS-Human Neurokinin 3 Receptor-HSV-Myc (SEQ ID NO:21).

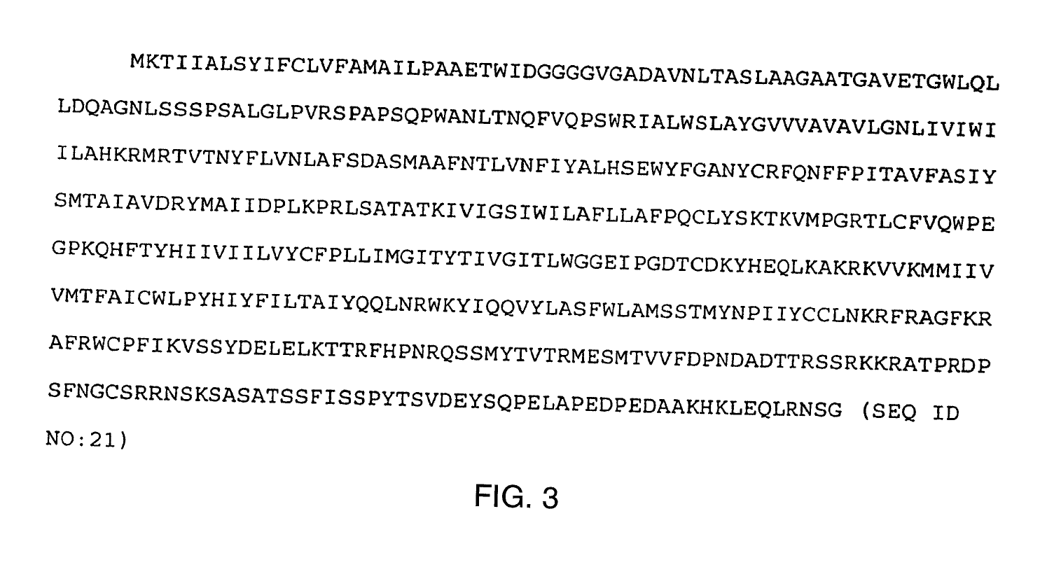

FIG. 4 presents the amino acid sequence of the HP protein Mellitin-Flag Tag-Human m1 mAChR-EE (SEQ ID NO:22).

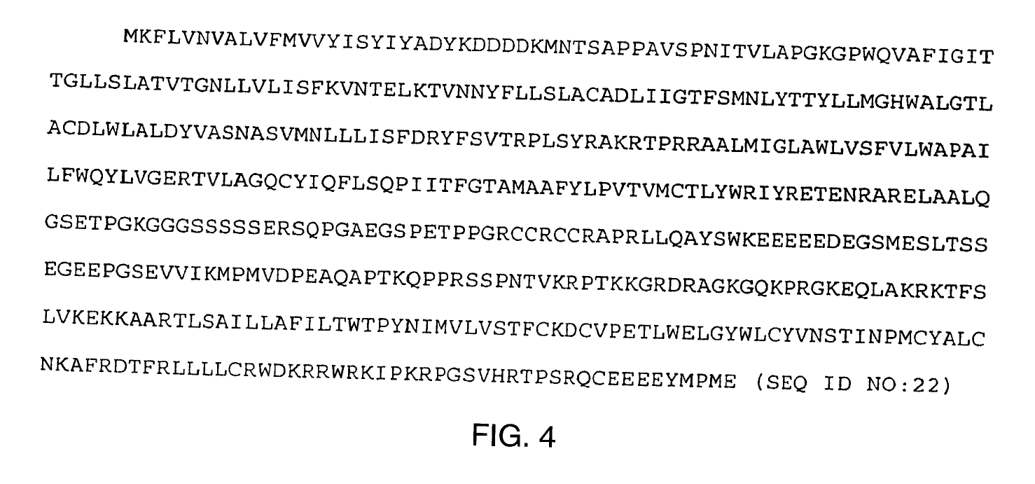

FIG. 5 presents the amino acid sequence of the HP protein Hemagglutinin SS-Rat m3 mAChR-HSV-OctaHis (SEQ ID NO:23).

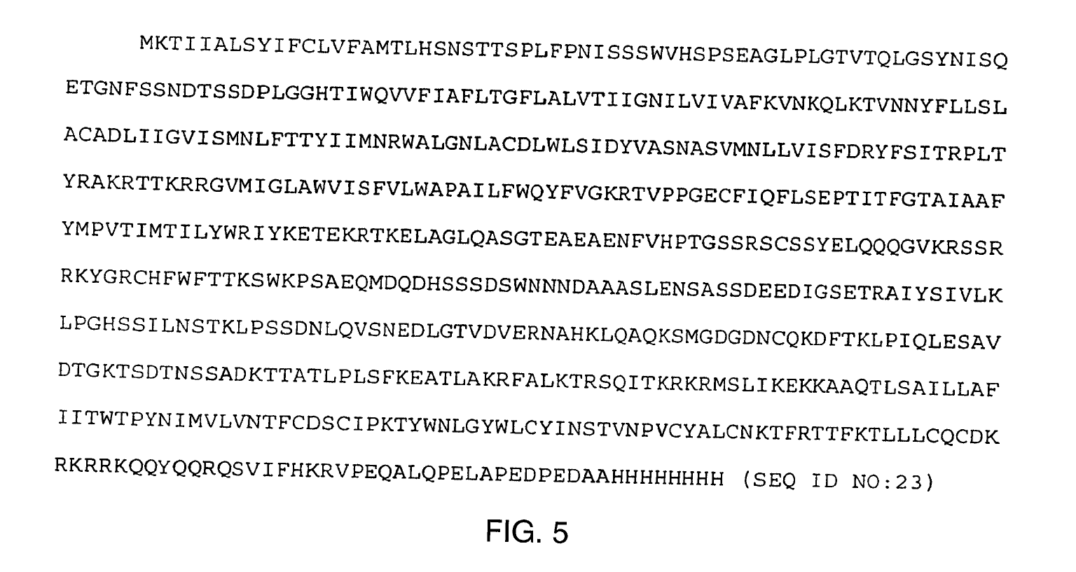

FIG. 6 presents SEC chromatograms represented by a screenshot from a computer interface developed to monitor the performance of the ALIS screening system. Relative absorbance at 230 nm is plotted on the vertical axis versus elution time following sample injection after injection onto the SEC column. The view compiles separation profiles for the analysis of six different binding reaction mixtures. In each case the mixture is composed of 25 :M each indomethacin and meclofenamate, 10 :M COX-1, and 1 :M each for approximately 2500 individual screening compounds. The peaks eluting between 12-15 seconds correspond to the COX-1 containing SEC fractions that are sent to the mass spectrometer for analysis. The peaks eluting after 17 seconds corresponds to unbound library members.

FIG. 7 presents mass spectral analysis showing the estimated recovery of two known COX-1 ligands (NSAID LM, composed of inclomethacin and meclofenamate) extracted from test libraries as described in FIG. 1. Different COX-1 preparations from different days (10/15 and 10/18) bind the known ligands in the absence and presence of competing libraries (NGL-15-A-137, NGL-10-A-41, NGL-116-A-470, NGM-51, NGM-108, NGM-177). By comparison to standard curves, the mass spectral analysis permits estimation of the pmol of each ligand recovered. Estimates were performed in triplicate for both indomethacin and meclofenamate.

FIG. 8 presents the structure of an example COX-1 ligand identified by ALIS. This compound, termed NGL-177-A-1128-A-2a, is one example of a compound identified as a COX-1 ligand by ALIS screening.

FIG. 9 presents a bar graph demonstrating competition with meclofenamate for COX-1 binding. Selected COX-1 hit compounds, each present at approximately 1 :M and identified by ten character names prefixed with NGL-x, were individually tested to determine whether they compete with 25 :M meclofenamate for binding to COX-1. The mass spectral response corresponding to the mass of either meclofenamate or the test ligand was quantified. For test ligands that are competitive with meclofenamate for COX-1 binding, the “ligand+competitor” response will be lower that the “ligand−competitor” response. Also, the meclofenamate response will be lower for that “ligand+competitor” trial than in the “COX1+25 :M Meclofenamate trial.”For example, the test compound represented by NGL-169-A-1151-A-4 is competitive with meclofenamate while the test compound represented by NGL-175-A-1127-A-1 is not significantly competitive.

FIG. 10 presents a bar graph demonstrating the extent of M2R1 ligand recovery quantified by the signal strength of the mass spectral analysis in accordance with the ALIS procedure. The x-axis is in relative units of mass spectrometric signal response for the respective masses of pirenzepine, QNB, and atropine.

### DETAILED DESCRIPTION OF THE PREFERRED EMBODIMENTS

The invention relates to the fields of pharmacology and medicine. More specifically, the invention relates to the screening of hydrophobic proteins for the identification of the respective ligand molecules with particular relevance to the development of novel medicines and medical diagnostics.

The patent and scientific literature cited herein establishes the knowledge that is available to those with skill in the art. The issued U.S. patents, allowed applications, published foreign applications, and references cited herein are hereby incorporated by reference. Any conflicts between these sources and the present specification shall be resolved in favor of the latter.

The invention provides an affinity selection-based HP screening method that can operate in the presence of an amphiphile without regard to the specific biological function of the HP target.

Aspects of the invention utilize techniques and methods common to the fields of molecular biology, cell biology and immunology. Useful laboratory references for these types of methodologies are readily available to those skilled in the art. See, for example, Molecular Cloning, A Laboratory Manual, 2nd. edition, edited by Sambrook, J., Fritsch, B. F. and Maniatis, T., (1989), Cold Spring Harbor Laboratory Press; Current Protocols In Molecular Biology and Current Protocols in Immunology, Wiley Interscience, New York; Harlow & Lane, Antibodies: A Laboratory Manual, Cold Spring Harbor Press, Cold Spring Harbor, N.Y. (1988).

The invention herein relates to the preparation and purification of hydrophobic proteins and to methods for the identification of ligands that bind specifically to hydrophobic proteins. As used herein, the term “hydrophobic protein” refers to any purified protein that is prepared with amphiphile. Alternatively, the term may also refer to any protein for which, when purified to greater than 1% purity or purified to greater than 10% purity or purified to greater than 25% purity or purified to greater than 50% purity, either requires or benefits from the presence of an amphiphile for functional assays, to enhance stability (shelf-life or ability to withstand freeze-thaw cycles), or to retain conformational integrity as observed by common laboratory techniques including enzymatic analysis, ligand binding assays, circular dichroism, hydrodynamic assessments of mean size, shape, or density, interaction with conformation-specific antibodies. In a preferred embodiment the hydrophobic protein of the invention is a mammalian hydrophobic protein. In a particularly preferred embodiment, the hydrophobic protein of the invention is a human hydrophobic protein.

The term “hydrophobic protein” is also meant to include proteins identified by bioinformatics-assisted means through the use of the following non-limiting examples of algorithms designed for the identification of hydrophobic proteins: (a) DAS—Prediction of transmembrane regions in prokaryotes using the Dense Alignment Surface method (Stockholm University) M. Cserzo, E. Wallin, I. Simon, G. von Heijne and A. Elofsson: Prediction of transmembrane alpha-helices in prokaryotic membrane proteins: the Dense Alignment Surface method; Prot. Eng. vol. 10, no. 6, 673-676,1997;(b) HMMTOP—Prediction of transmembrane helices and topology of proteins (Hungarian Academy of Sciences) G. E Tusnády and I. Simon (1998) Principles Governing Amino Acid Composition of Integral Membrane Proteins: Applications to Topology Prediction. J. Mol. Biol. 283, 489-506; (c) Hidden Markov Model Predictions E L L Sonnhammer, G. von Heijne, and A. Krogh: A hidden Markov model for predicting transmembrane helices in protein sequences. Proc. of the Sixth Intern Conf. on Intelligent Systems for Molecular Biology (ISMB98), 175-182, 1998; (D) TMAP—Transmembrane detection based on multiple sequence alignment (Karolinska Institut; Sweden) No reference available: see URL at http//www.mbb.ki.se/tmap/; and (e) TopPred 2—Topology prediction of membrane proteins (Stockholm University). “Membrane Protein Structure Prediction, Hydrophobicity Analysis and the Positive-inside Rule”, Gunnar von Heijne. J. Mol. Biol. (1992) 225, 487-494 and M. Cserzo, E. Wallin, I. Simon, G. von Heijne and A. Elofsson: Prediction of transmembrane alpha-helices in prokaryotic membrane proteins: the Dense Alignment Surface method; Prot. Eng. vol. 10, no. 6, 673-676,1997.

For a comparison of these methods see “Prediction of transmembrane alpha-helices in prokaryotic membrane proteins: the dense alignment surface method”, Miklos Cserzo, Erik Wallin, Istvan Simon, Gunnar von Heijne, and Arne Elofsson, to appear in Protein Engineering, vol. 10, no. 6, (1997).

For exemplary purposes only, non-limiting examples of hydrophobic proteins (as the term is used herein) are presented in Table 1. These proteins are listed in GenBank, as indicated by the locus designations from the National Center for Biotechnology Information (NCBI).

TABLE 1
Non-Limiting Examples of Hydrophobic Proteins

| Common Name | NCBI Locus |
|---|---|
| Kvl.3 | LOCUS NP_002223 523 aa PRI |
| Shaker Family K⁺ Channel | 31 Oct. 2000 DEFINITION |
| Polytopic | potassiumvoltag e-gated |
| | channel, shaker-related |
| | subfamily, member 3 [Homo |
| | sapiens]. ACCESSION |
| | NP_002223 |
| | PID g4504815 |
| | VERSION NP_002223.1 |
| | GI:4504815 |
| m2 Muscarinic Acetylcholine | LOCUS NP 000730 466 aa PRI |
| Receptor | 31 Oct. 2000 DEFINITION |
| G Protein-Coupled Receptor | cholinergic receptor, |
| Class A, Polytopic | muscarinic 2; muscarinic |
| | acetylcholine receptor M2 |
| | [Homo sapiens]. ACCESSION |
| | NP_000730 |
| | PID g4502817 |
| | VERSION NP_000730.1 |
| | GI:4502817 |
| | DBSOURCE REFSEQ: accession |
| | NM 000739.1 |
| Secretin Receptor | LOCUS NP 002971 440 aa PRI |
| G Protein-Coupled Receptor | 31 Oct. 2000 |
| Class B, Polytopic | DEFINITION secretin receptor |
| | [Homo sapiens]. |
| | ACCESSION NP_002971 |
| | PID g4506825 |
| | VERSION NP_002971.1 |
| | GI:4506825 |
| | DBSOURCE REFSEQ: accession |
| | NM 002980.1 |
| Metabotropic Glutatmate | LOCUS NP 000832 912 aa PRI |
| Receptor, Type 4 | 31 Oct. 2000 |
| G Protein-Coupled Receptor | DEFINITION glutamate |
| Class C, Polytopic | receptor, metabotropic 4 |
| | [Homo sapiens]. ACCESSION |
| | NP_000832 |
| | PID g4504141 |
| | VERSION NP_000832.1 |
| | GI:4504141 |
| | DBSOURCE REFSEQ: accession |
| | NM 000841.1 |
| Epidermal Growth Factor | LOCUS AAB19486 10 aa PRI |
| Receptor | 29 Jun. 2000 |
| Transmembrane Receptor Kinase | DEFINITION epidermal growth |
| | factor receptor; EGFR [Homo |
| | sapiens]. ACCESSION AAB19486 |
| | PID g8815559 |
| | VERSION AAB19486.2 |
| | GI:8815559 |
| | DBSOURCE locus S51343 |
| | accession |
| | S51343.1 |
| Cyclooxygenase-2 (COX-1) | LOCUS PGH2_HUMAN 604 aa |
| Integral Membrane Enzyme | PRI 15 Dec. 1998 |
| Monotopic | DEFINITION PROSTAGLANDIN |
| | G/H SYNTHASE 2 PRECURSOR |
| | (CYCLOOXYGENASE-2) (COX-1) |
| | (PROSTAGLANDIN-ENDOPER- |
| | OXIDE SYNTHASE 2) (PROSTA- |
| | GLANDIN H2 SYNTHASE 2) |
| | (PGH SYNTHASE 2) (PGHS-2) |
| | (PHS II). |
| | ACCESSION P35354 |
| | PID g3915797 |
| | VERSION P35354 GI:3915797 |
| | DBSOURCE swissprot: locus |
| | PGH2 HUMAN, accession P35354 |
| Ca⁺⁺ ATPase | LOCUS NP_001675 1205 aa |
| Integral Membrane Enzyme | PRI 31 Oct. 2000 |
| Polytopic | DEFINITION ATPase, Ca++ |
| | transporting, plasma membrane |
| | 4 [Homo sapiens]. ACCESSION |
| | NP_001675 |
| | PID g4502289 |
| | VERSION NP_001675.1 |
| | GI:4502289 |
| | DBSOURCE REFSEQ: accession |
| | NM_001684.1 |
| | EC 3.6.1.38 |
| Cytochrome c Oxidase | 13 Distinct Polypeptide |
| Integral Membrane Enzyme | Subunits |
| Polytopic | See Protein Data Bank #10CC |
| | for details. |
| | http://www.rcsb.org/pdb/cgi/e |
| | xplore.cgi?job=chains&pdbId=1 |
| | OCC&page=&pid=4725 |
| | EC 1.9.3.1 |
| Aquaporin, Type 3 | LOCUS NP 004916 292 aa PRI |
| Channel, Polytopic | 01 Nov. 2000 |
| | DEFINITION aquaporin 3 [Homo |
| | sapiens]. ACCESSION |
| | NP_004916 |
| | PID g4826645 |
| | VERSION NP_004916.1 |
| | GI:4826645 |
| | DBSOURCE REFSEQ: accession |
| | NM 004925.2 |
| Outer Membrane Phospholipase | See Protein Data Bank #1QD5 |
| A | for details. EC 3.1.1.32 |
| Integral Membrane Enzyme | |
| Polytopic [symbol] -Barrel | |
| Serotonin Transporter | LOCUS NP 001036 630 aa PRI |
| Transporter, Topology Unknown | 31 Oct. 2000 |
| | DEFINITION solute carrier |
| | family 6 (neurotransmitter |
| | transporter, serotonin), |
| | member 4; Solute carrier |
| | family 6 (neurotransmitter |
| | transporter, serotonin) |
| | [Homo sapiens] |
| | ACCESSION NP_001036 |
| | PID g4507043 |
| | VERSION NP_001036.1 |
| | GI:4507043 |
| DBSOURCE REFSEQ: accession | |
| | NM 001045.1 |
| Erythropoietin Receptor | LOCUS NP 058698 507 aa ROD |
| Non-Enzymatic Transmembrane | 01 Nov. 2000 |
| Receptor | DEFINITION erythropoietin |
| | receptor [Rattus norvegicus]. |
| | ACCESSION NP_058698 |
| | PID g8393319 |
| | VERSION NP_058698.1 |
| | GI:8393319 |
| | DBSOURCE REFSEQ: accession |
| | NM 017002.1 |

As will be understood by those in the art, a method of identifying a ligand for a hydrophobic protein is synonymous with a method of screening for a ligand that binds a small molecule. Furthermore, as used herein, the term “screening” refers to a procedure used to detect the interaction between polypeptide, for example a hydrophobic protein, and a small molecule; it is useful for discriminating between ligands that bind to proteins with a Kd<200 μM from large ensembles of ligands that either do not bind to the protein or bind only weakly with a Kd>200 μM.

The present invention utilizes mass spectrometry (MS) in the identification of hydrophobic protein ligands. The MS technique is only rarely performed to analyze samples containing hydrophobic proteins because these samples contain detergent amphiphiles. Detergents suppress analyte ion formation, a critical phenomenon for MS, and so significantly hamper MS, that reports of successful MS analysis of hydrophobic proteins are few. Nevertheless, several labs have tried to perform MS analysis of purified hydrophobic proteins by identifying methods to remove the detergent prior to MS analysis. All of these labs use matrix-assisted laser desorption ionization (MALDI) to present the protein sample to the detector.

However, all of the known methods for sample preparation of hydrophobic proteins use organic solvents and/or acid to extract the detergent from the polypeptide prior to MS analysis. Such treatment denatures the polypeptide, a fact that precludes the binding of ligands to the analyte hydrophobic protein. For researchers interested in using MS for the study of hydrophobic protein-ligand interactions, denaturing preparation methods are not suitable. Moreover, the preparation of a membrane protein for analysis by MALDI-MS is laborious compared to the method provided by the present invention.

By contrast, in certain preferred embodiments the present invention uses electrospray ionization (ESI) MS which permits the fluid handling of a membrane protein sample as it passes from the SEC separation to the MS detector. As such, the MS analysis proceeds in less than 30 seconds after the sample is sent to the RPC column.

The reasons why detergents must be removed from protein samples prior to mass spectrometric analysis are well known, and are provided, e.g., in the following references: (a) BioTechniques (1997) 22:244-250; J. P C. Vissers, J. -P Chervet K. Sanborn, and J. -P Salzmann; (b) Protein Science (1994) 3:1975-1983; R. R. Ogorzalek Loo, N. Dales and P. C. Andrews; (c) J. Mass. Spectrom. (1995) 30:1462-8. Rosinke B, Strupat, K, et al.; Methods Enzymol. (1996) 270:519-51; Beavis, R C and Chait, B T; and Proc. Natl. Acad. Sci. USA (1990) 87:6873-7; Beavis, R C and Chait, B T.; Fearnley, I. M. et a;., Biochem. Soc. Trans., (1996) 24:12-917.

In a first aspect, the invention provides a method for identifying a ligand for a hydrophobic protein, the method comprising (a) selecting a ligand molecule by affinity selection by exposing a hydrophobic target protein bound by an amphiphile to a multiplicity of molecules to promote the formation of at least one complex between the hydrophobic target protein and the ligand molecule; (b) separating the complex from the unbound molecules; and (c) identifying the ligand molecule.

The affinity screening methods of the invention are to be distinguished from functional selection methodologies. Functional selection methodologies involve ligand selection based on criteria that identify some specific protein-ligand or protein-protein interaction as significant; such ligand selection depends on either an action assignable to the protein (e.g., chemical catalysis for enzymes) or identification of some interaction between the protein and some other molecule (e.g., interaction between a protein and a known small molecule ligand in the case of non-enzymes). In summary, these screening methodologies are generally based on enzymatic or biofunctional assays or ligand displacement assays.

Various embodiments of the method of the invention may utilize an automated ligand identification system (ALIS) for the discovery of novel drug leads. ALIS selects ligands based on the affinity of the compound for its target protein and identifies the ligands by mass spectrometry (see International Patent Application WO 99/35109). The invention herein provides for the application of ALIS to amphiphile complexed HPs.

As used herein, the term “affinity selection” means a ligand selection based on the affinity of one molecule for a selected protein target; such ligand selection is independent of the functional activity of the protein of interest other than for the fact the protein binds the small molecule.

As used herein, the term “amphiphile” is used to mean any molecule generally with the properties of a detergent, phospholipid, or surfactant that enhances the water solubility of hydrophobic polypeptides; specifically any molecule known to assume an association colloid in aqueous solution; non-limiting examples of such amphiphiles would include phospholipids and other polar lipids (exemplified by phosphatidylcholines, lysophospholipids, cholesterols, lecithins, ceramides, etc); amphiphilic macromolecular polymers (exemplified by the work of Christophe Tribet and Jean-Luc Popot (Tribet, C. et al. J. L. Natl. Acad. Sci. USA (1996) 93:15047-50); surfactants including alkyl saccharides, alkyl thioglycosides, alkyl dimethylamine oxides, bile acid derivatives like cholate and the CHAPS series, FOS-CHOLINE™ series, CYMAL™ or CYGLU™ series, glucamides, and alkyl polyoxyethylenes, etc; or polypeptides known to adopt amphipathic structures (exemplified by work of C. E. Schafmeister and R. M. Stroud (Schafmeister, C. E. et al., RM Science (1993) 262:734-8).

As used herein, the term “multiplicity of molecules” refers to a plurality of molecules to be tested for the property of specific binding to a hydrophobic target protein. By the term “molecule” is meant, any compound in the size range of 150 to 5000 atomic mass units (amu). Such compounds may be generated by any means known in the art. Particularly favored methods of generating the multiplicity of molecules is through the use of combinatorial chemistry. A “combinatorial library” refers to a plurality of molecules or compounds which are formed by combining, in every possible way for a given compound length, a set of chemical or biochemical building blocks which may or may not be related in structure. Alternatively, the term can refer to a plurality of chemical or biochemical compounds which are formed by selectively combining a particular set of chemical building blocks. For example, twenty amino acids randomly combined into hexameric peptides will produce no less than 64 million compounds. With the “combinatorial library” approach, as many different compounds as possible are made, and then candidate compounds are selected by screening them for binding activity against the target molecule of interest, e.g., a hydrophobic protein.

There are now many methods well known in the art for the construction of combinatorial libraries. By way of non-limiting example, the following references provide methods for combinatorial library construction: U.S. Pat. No. 6,147,344; (WO 99/35109; U.S. Pat. Nos. 6,114,309; 6,025,371; 6,017,768; 5,962,337; 5,919,955; and 5,856,496, to name a few. As used herein, the term “multiplicity of molecules” may also refer to a natural plurality of molecules or compounds, obtained for example from body fluids, tissues or cells. These samples may be manipulated, e.g., proteolytically digested, in vitro prior to their use in a ligand screening protocol.

In certain embodiments of the first aspect, exposure of the hydrophobic target protein to a multiplicity of molecules occurs under homogeneous solution phase conditions. In certain embodiments of the first aspect, exposure of the hydrophobic target protein to a multiplicity of molecules occurs under heterogeneous solution phase conditions. In certain embodiments of the first aspect, selection of the ligand molecule is done using multi-dimensional chromatography.

As used herein, the term “homogeneous solution phase” means a protein preparations whereby a protein is combined with (a) ligand(s) with the intent to facilitate possible protein-ligand interactions; such preparations are found as sols in the temperature range of −40 to 60° C. such that neither protein nor ligand are bound to a supporting element; these preparations would either pass through a semipermeable membrane with a size cut-off of 5.0 μM or behave as though they a sedimentation coefficient of less than 500 Svedbergs or both; examples of such preparations would include combinations of ligand(s) with proteins that are solubilized in amphiphile, proteins incorporated into proteoliposomes, proteins incorporated into cell-derived virus-like particles, etc.

As used herein, the term “heterogeneous solution phase” means a protein preparations whereby a protein is combined with (a) ligand(s) with the intent to facilitate possible protein-ligand interactions; such preparations are found as mixtures in the temperature range of −40 to 60° C. such that either the protein or the ligand is bound to a supporting element; these preparations would either fail to pass through a semipermeable membrane with a size cut-off of 5.0 μM or they would behave as though they have a sedimentation coefficient of greater than 500 Svedbergs or both; examples of such preparations would include combinations of ligand(s) with proteins that are presented on the surface of a bead/stationary element whereby the protein is attached to the bead/stationary element through either a covalent or non-covalent linkage or a linkage dependent upon the self-association of amphiphiles or combinations of proteins with ligands that are fixed to a stationary phase.

As used herein, the term “multi-dimensional chromatography” means a procedure for processing a sample involving more than one chromatographic method in tandem. Representative types of chromatographic methods include: (1) solid phase chromatography media: any of a variety of materials including small particles (<5 μM), solid porous castings, filters, or semipermeable membranes that may commonly be referred to as resins, gels, immobilized artificial membranes, stationary phase elements, or otherwise, and are used with the intent of providing stationary surfaces over which or through which solubilized analytes are passed or with which solubilized analytes interact as in chromatographic or electrophoretic separations or fractionations; and (2) solution phase chromatography media: solutions or fluids suitable for use in electrophoretic separations or fractionations when used in combination with stationary phase chromatography media.

In certain embodiments of the first aspect, the hydrophobic target protein is selected from the group consisting of a membrane protein, an integral membrane protein, a transmembrane protein, a monotopic membrane protein, a polytopic membrane protein, a pump protein, a channel protein, a receptor kinase protein, a G protein-coupled receptor protein, a membrane-associated enzyme, and a transporter protein.

In certain embodiments of the first aspect, the multiplicity of molecules is a mass coded library of molecules. In certain embodiments of the first aspect, the multiplicity of molecules is a library of molecules that is not mass coded.

As used herein, the term “mass-coded library” refers to a mass coded combinatorial library. The compounds of the mass-coded combinatorial library are of the general formula X(Y)ₙ, wherein X is a scaffold, each Y is a peripheral moiety and n is an integer greater than 1, typically from 2 to about 6. The term “scaffold”, as used herein, refers to a molecular fragment to which two or more peripheral moieties are attached via a covalent bond. The scaffold is a molecular fragment which is common to each member of the mass-coded set of compounds. The term “peripheral moiety”, as used herein, refers to a molecular fragment which is bonded to a scaffold. Each member of the set of mass-coded compounds will include a combination of n peripheral moieties bonded to the scaffold and this set of compounds forms a mass-coded combinatorial library. More details of mass-coded libraries are provided in the patent application WO9935109A1, which is incorporated herein by reference.

As used herein, the phrase “a library of molecules that is not mass-coded” means any plurality of molecules or compounds that are not produced by a mass-coded combinatorial process. Thus, the term includes any and all other methods of producing a combinatorial library. In addition, the term also includes compounds constructed by “Structure Based Drug Design” methodology, which seeks to design a drug based on the structure of the target protein, and natural libraries of compounds obtained from body fluids, tissues or cells.

In certain embodiments of the first aspect, the amphiphile is selected from the group consisting of (a) a polar lipid, (b) an amphiphilic macromolecular polymer, (c) a surfactant or detergent, and (d) an amphiphilic polypeptide. In certain embodiments of the first aspect, ligand identification is done by mass spectral analysis. In certain embodiments of the first aspect, the ligand molecule is deconvoluted by mass spectral analysis. In certain embodiments of the first aspect, separation of the complex from the unbound molecules is accomplished with solid phase chromatography media.

By the term “ligand identification” as used herein is meant any process that can accurately specify the structural composition of a small molecule detected in a screen.

In certain embodiments of the first aspect, the hydrophobic target protein comprises (a) at least one transmembrane domain sequence, (b) at least two tag sequences useful for affinity selection, and (c) a hydrophobic protein (HP) sequence. In certain embodiments thereof, the hydrophobic protein sequence is selected from the group consisting of (a) a membrane protein, (b) an integral membrane protein, (c) a transmembrane protein, (d) a monotopic membrane protein, (e) a polytopic membrane protein, (f) a pump protein, (g) a channel protein, (h) a receptor kinase protein, (i) a G protein-coupled receptor protein, (j) a membrane-associated enzyme, and (k) a transporter protein. In certain embodiments thereof, the tag sequences comprise epitope tag sequences selected from the group consisting of (a) a FLAG tag (NH2-DYKDDDDK-COOH) (SEQ ID NO:1), (b) an EE tag (NH2-EEEEYMPME-COOH) (SEQ ID NO:2), (c) a hemagglutinin tag (NH2-YPYDVPDYA-COOH) (SEQ ID NO:3), (d) a myc tag (NH2-KHKLEQLRNSGA-COOH) (SEQ ID NO:4), (e) an HSV tag (NH2-QPELAPEDPED-COOH) (SEQ ID NO:5) and (f) a rhodopsin tag (NH2GTEGPNFYVPFSNKTGVVRSPFEAPQYYLAEPWQFSM-COOH) (SEQ ID NO:6).

In certain embodiments of the first aspect, the hydrophobic target protein comprises a sequence with an amino terminus to carboxy terminus order selected from the group consisting of (a) Tag1-Tag2-HP, (b) Tag1-HP-Tag2, and (c) HP-Tag1-Tag2. In certain embodiments of thereof, the invention provides a method wherein the hydrophobic target protein is selected from the group consisting of (a) Myc tag-EE tag-Human m2 mAChR (SEQ ID NO:6), (b) Flag tag-Human Beta 2 Adrenergic Receptor-EE tag (SEQ ID NO:7), (c) Human Neurokinin 3 Receptor-HSV tag-Myc tag (SEQ ID NO:9), (d) Flag tag-Human m1 mAChR-EE tag (SEQ ID NO:10), and (e) Rat m3 mAChR-HSV tag-OctaHis tag (SEQ ID NO:11). In certain embodiments thereof, the invention provides a method wherein the hydrophobic target protein further comprises a heterologous signal sequence (SS) at the amino terminus. In certain embodiments thereof, the heterologous signal sequence is selected from the group consisting of (a) the Mellitin signal sequence of NH₂-KFLVNVALVFMVVYISYIYA-COOH (SEQ ID NO:12), (b) the GP signal sequence of NH₂-VRTAVLILLLVRFSEP-COOH (SEQ ID NO:13), (c) the Hemagglutinin signal sequence of NH₂-KTIIALSYIFCLVFA-COOH (SEQ ID NO:14), (d) the rhodopsin tag 1 signal sequence of NH₂-MNGTEGPNFYVPFSNKTGVVRSPFEAPQYYLAEP-COOH (SEQ ID NO:15), and (e) the rhodopsin tag ID4 signal sequence of NH₂-GKNPLGVRKTETSQVAPA-COOH (SEQ ID NO:16). In certain embodiments thereof the tag sequences further comprise a hexahistidine sequence (SEQ ID NO:17) and a decahistidine sequence (SEQ ID NO:18). In yet certain embodiments thereof the hydrophobic target protein is selected from the group consisting of (a) GP67 SS-Myc tag-EE tag-Human m2 mAChR (SEQ ID NO:19), (b) Mellitin SS-Flag tag-Human Beta 2 Adrenergic Receptor-EE tag(SEQ ID NO:20), (c) Hemagglutinin SS-Human Neurokinin 3 Receptor-HSV tag-Myc tag (SEQ ID NO:21), (d) Mellitin SS-Flag tag-Human m1 mAChR-EE tag (SEQ ID NO:22), and (e) Hemagglutinin SS-Rat m3 mAChR-HSV tag-OctaHis tag (SEQ ID NO:23).

In a second aspect, the invention provides a method of isolating a hydrophobic protein, the method comprising (a) purifying the hydrophobic protein by sucrose gradient ultracentrifugation, (b) purifying the hydrophobic protein by antibody affinity purification, and (c) purifying the hydrophobic protein by immobilized metal affinity chromatography.

In certain embodiments of the second aspect, the hydrophobic protein comprises (a) at least one transmembrane domain sequence, (b) at least two tag sequences useful for affinity selection, and (c) a hydrophobic protein (HP) sequence. In certain embodiments thereof, the hydrophobic protein sequence is selected from the group consisting of (a) a membrane protein, (b) an integral membrane protein, (c) a transmembrane protein, (d) a monotopic membrane protein, (e) a polytopic membrane protein, (f) a pump protein, (g) a channel protein, (h) a receptor kinase protein, (i) a G protein-coupled receptor protein, (j) a membrane-associated enzyme, and (k) a transporter protein. In certain embodiments of the second aspect, the tag sequences of the hydrophobic protein comprise epitope tag sequences selected from the group consisting of (a) a FLAG tag (NH2-DYKDDDDK-COOH) (SEQ ID NO:1), (b) an EE tag (NH2-EEEEYMPME-COOH) (SEQ ID NO:2), (c) a hemagglutinin tag (NH2-YPYDVPDYA-COOH) (SEQ ID NO:3), (d) a myc tag (NH2-KHKLEQLRNSGA-COOH) (SEQ ID NO:4), (e) an HSV tag (NH2-QPELAPEDPED-COOH) (SEQ ID NO:5) and (f) a rhodopsin tag (NH2 MNGTEGPNFYVPFSNKTGVVRSPFEAPQYYLAEPWQFSM-COOH) (SEQ ID NO:6). In certain embodiments of the second aspect, the hydrophobic protein comprises a sequence with an amino terminus to carboxy terminus order selected from the group consisting of (a) Tag1-Tag2-HP, (b) Tag1-HP-Tag2, and (c) HP-Tag1-Tag2.

In certain embodiments of the second aspect, the hydrophobic protein is selected from the group consisting of (a) Myc tag-EE tag-Human m2 mAChR (SEQ ID NO:7), (b) Flag tag-Human Beta 2 Adrenergic Receptor-EE tag (SEQ ID NO:8), (c) Human Neurokinin 3 Receptor-HSV tag-Myc tag (SEQ ID NO:9), (d) Flag tag-Human m1 mAChR-EE tag (SEQ ID NO:10), and (e) Rat m3 mAChR-HSV tag-OctaHis tag (SEQ ID NO:11). In embodiments thereof, the hydrophobic protein further comprises a heterologous signal sequence (SS) at the amino terminus. In certain embodiments thereof, the heterologous signal sequence is selected from the group consisting of (a) the Mellitin signal sequence of NH2-KFLVNVALVFMVVYISYIYA-COOH (SEQ ID NO:12), (b) the GP signal sequence of NH2-VRTAVLILLLVRFSEP-COOH (SEQ ID NO:13), (c) the Hemagglutinin signal sequence of NH2-KTIIALSYIFCLVFA-COOH (SEQ ID NO:14), (d) the rhodopsin tag 1 signal sequence of NH2-MNGTEGPNFYVPFSNKTGVVRSPFEAPQYYLAEP-COOH (SEQ ID NO:15), and (e) the rhodopsin tag ID4 signal sequence of NH2-GKNPLGVRKTETSQVAPA-COOH (SEQ ID NO:16). In certain embodiments of the second aspect, the tag sequences of the hydrophobic protein further comprise a hexahistidine sequence (SEQ ID NO:17) and a decahistidine sequence (SEQ ID NO:18).

In certain embodiments of the second aspect, the hydrophobic target protein is selected from the group consisting of (a) GP67 SS-Myc tag-EE tag-Human m2 mAChR (SEQ ID NO:19), (b) Mellitin SS-Flag tag-Human Beta 2 Adrenergic Receptor-EE tag(SEQ ID NO:20), (c) Hemagglutinin SS-Human Neurokinin 3 Receptor-HSV tag-Myc tag (SEQ ID NO:21), (d) Mellitin SS-Flag tag-Human m1 mAChR-EE tag (SEQ ID NO:22), and (e) Hemagglutinin SS-Rat m3 mAChR-HSV tag-OctaHis tag (SEQ ID NO:23).

In a third aspect, the invention provides an isolated nucleic acid molecule suitable for hydrophobic protein expression, comprising (a) a vector polynucleotide sequence for protein expression in a eukaryotic cell, and (b) a polynucleotide sequence encoding an engineered hydrophobic protein comprising the following elements (i) an N-terminal methionine residue, (ii) a heterologous signal sequence (SS), (iii) at least one transmembrane domain sequence, (iv) at least two tag sequences useful for affinity purification, and (v) a hydrophobic protein (HP) sequence.

By the phrase “a vector polynucleotide sequence for protein expression in a eukaryotic cell” is meant any polynucleotide sequence comprising an origin of replication allowing replication in a eukaryotic cell, a selectable marker, e.g., antibiotic resistance marker, and a promoter sequence element to promote transcription of the structural gene, which may be viral, prokaryotic or eukaryotic in origin. The origin of the vector polynucleotide sequence may be viral, prokaryotic or eukaryotic or a combination thereof. As will be understood in the art, the vector sequence while being designed for expression in a eukaryotic cell may optionally contain a prokaryotic origin of replication. Non-limiting examples of suitable vector polynucleotide sequences include the following: baculovirus vectors such as pVL1392 (Pharmingen, San Diego, Calif.) and pBAC-1 (Novagen, Madison, Wis.) and mammalian expression vectors such as pcDNA 3.1 (Invitrogen, San Diego, Calif.) and pTriEx-1 (Novagen, Madison, Wis.).

The appropriate DNA sequence may be inserted into the vector by a variety of procedures. In general, the DNA sequence is inserted into an appropriate restriction endonuclease site(s) by procedures known in the art. Such procedures and others are deemed to be within the scope of those skilled in the art.

In certain embodiments, the present invention relates to host cells containing the above-described constructs. The host cell can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by any means known in the art, including but not limited to transduction or transformation or transfection or electroporation.

Examples of a suitable heterologous signal sequences (SS) include the honey bee mellitin SS (NH2-MKFLVNVALVFMVVYISYIYA-COOH) (SEQ ID NO:12) (Tessier D.C., (1991) Gene 98: 177), see also Invitrogen.com's pMelBac product), the baculovirus gp67 SS (NH2-MVRTAVLILLLVRFSEP-COOH) (SEQ ID NO: 13) (Kretzschmar T., et al., (1996) J. Immunol Methods 195: 93-101), the Influenza A virus hemagglutinin SS (NH2-MKTIIALSYIFCLVFA-COOH) (SEQ ID NO:14) (Verhoeyen, M., (1980) Nature 286: 771-776), the rhodopsin tag 1 SS (NH2-MNGTEGPNFYVPFSNKTGVVRSPFEAPQYYLAEP-COOH) (SEQ ID NO:15), or the rhodopsin tag ID4 SS (NH2-GKNPLGVRKTETSQVAPA-COOH) (SEQ ID NO:16). Generally, such suitable SSs will be less that 75 aa long. These signal sequences may or may not be cleaved from the protein upon expression in animal cells. For example, the rhodopsin tag 1 is not cleaved off of the protein when presented by the cell to the plasma membrane, whereas rhodopsin tag ID4 signal sequence is cleaved off.

Non-limiting examples of a suitable epitope affinity tags include FLAG (NH2-DYKDDDDK-COOH) (SEQ ID NO:29), “EE” (NH2-EEEEYMPME-COOH) (SEQ ID NO:30), hemagglutinin (NH2-YPYDVPDYA-COOH) (SEQ ID NO:31), myc (NH2-KHKLEQLRNSGA-COOH) (SEQ ID NO:32), or herpes simplex virus tag (“HSV”; NH2-QPELAPEDPED-COOH) (SEQ ID NO:33).

These design elements of the hydrophobic protein are arrayed from amino to carboxy terminus in one of the following permutations: (1) SS-Tag1-Tag2-HP; (2) SS-Tag1-HP-Tag2; (3) SS-HP-Tag1-Tag2.

In certain embodiments thereof, the N-terminal methionine sequence and the heterologous signal sequence are selected from the group consisting of (a) MKFLVNVALVFMVVYISYIYA (SEQ ID NO:24), (b) MVRTAVLILLLVRFSEP (SEQ ID NO:25), (c) MKTIIALSYIFCLVFA (SEQ ID NO:26) (d) MMNGTEGPNFYVPFSNKTGVVRSPFEAPQYYLAEP-COOH (SEQ ID NO:27) and (e) MGKNPLGVRKTETSQVAPA-COOH (SEQ ID NO:28).

In certain embodiments thereof, the tag sequences comprise epitope tag sequences selected from the group consisting of (a) a FLAG tag (NH2-DYKDDDDK-COOH) (SEQ ID NO:1), (b) an EE tag (NH2-EEEEYMPME-COOH) (SEQ ID NO:2), (c) a hemagglutinin tag (NH2-YPYDVPDYA-COOH) (SEQ ID NO:3), (d) a myc tag (NH2-KHKLEQLRNSGA-COOH) (SEQ ID NO:4), (e) an HSV tag (NH2-QPELAPEDPED-COOH) (SEQ ID NO:5), and (f) a rhodopsin tag (NH2 MNGTEGPNFYVPFSNKTGVVRSPFEAPQYYLAEPWQFSM-COOH) (SEQ ID NO:6).

In certain embodiments of the third aspect, the elements of the engineered hydrophobic protein are arrayed from an amino to carboxy terminus order selected from the group consisting of (a) SS-Tag1-Tag2-HP, (b) SS-Tag1-HP-Tag2, and (c) SS-HP-Tag1-Tag2. In embodiments thereof, the engineered hydrophobic protein is selected from the group consisting of (a) GP67-Myc-EE-Human m2 mAChR (SEQ ID NO:19), (b) Mellitin-Flag Tag-Human Beta 2 Adrenergic Receptor-EE (SEQ ID NO:20), and (c) Hemagglutinin SS-Human Neurokinin 3 Receptor-HSV-Myc (SEQ ID NO:21). In a further embodiment of the third aspect, the tag sequences further comprise a hexahistidine sequence (SEQ ID NO:17) and a decahistidine sequence (SEQ ID NO:18). In certain embodiments of the third aspect, the engineered hydrophobic protein is selected from the group consisting of (a) GP67-Myc-EE-Human m2 mAChR (SEQ ID NO:19), (b) Mellitin-Flag Tag-Human m1 mAChR-EE (SEQ ID NO:20), and (c) Hemagglutinin SS-Rat m3 mAChR-HSV-OctaHis (SEQ ID NO:21).

The HP sequence of the isolated polynucleotide may be any polynucleotide encoding a protein that is isolated with amphiphile present. Alternatively, the term may also refer to any polynucleotide encoding a protein for which, when purified to greater than 1% purity or purified to greater than 10% purity or purified to greater than 25% purity or purified to greater than 50-99% purity, either requires or benefits from the presence of an amphiphile for functional assays, to enhance stability (shelf-life or ability to withstand freeze-thaw cycles), or to retain conformational integrity as observed by common laboratory techniques including ligand binding assays, circular dichroism, hydrodynamic assessments of mean size, shape, or density, interaction with conformation-specific antibodies. In a preferred embodiment the hydrophobic protein of the invention is a mammalian hydrophobic protein. In a particularly preferred embodiment, the hydrophobic protein of the invention is a human hydrophobic protein.

The HP sequence of the isolated polynucleotide may include polynucleotides encoding proteins that are identified by bioinformatics-assisted means through the use of the following non-limiting examples of algorithms designed for the identification of hydrophobic proteins: (a) DAS—Prediction of transmembrane regions in prokaryotes using the Dense Alignment Surface method (Stockholm University) Cserzo, M. et al. (1997) Prot. Eng. 10:673-676;(b) HMMTOP—Prediction of transmembrane helices and topology of proteins (Hungarian Academy of Sciences) G. E Tusnády and I. Simon (1998) J. Mol. Biol. 283: 489-506; (c) Hidden Markov Model Predictions Sonnhammer, E. L. L. et al. (1998) A hidden Markov model for predicting transmembrane helices in protein sequences. Proc. of the Sixth Intern. Conf. on Intelligent Systems for Molecular Biology (ISMB98), 175-182; (D) TMAP—Transmembrane detection based on multiple sequence alignment (Karolinska Institut; Sweden) No reference available: see URL at http//www.mbb.ki.se/tmap/; and (e) TopPred 2—Topology prediction of membrane proteins (Stockholm University). von Heijne, G. (1992) J. Mol. Biol. 225:487-494 and Cserzo, M. et al. (1997) Prot. Eng. 10:673-676.

Representative non-limiting examples of proteins that may be encoded by the HP polynucleotide of the invention are presented herein in Table 1.

All nucleic acid sequences and the respective amino acid sequences encoded thereby identified above by the appropriate GenBank accession numbers are herein incorporated by reference.

In a fourth aspect, the invention provides a method for identifying a ligand for a hydrophobic protein, the method comprising (a) selecting a hydrophobic target protein from the group consisting of (i) a membrane protein, (ii) an integral membrane protein, (iii) a transmembrane protein, (iv) a monotopic membrane protein, (v) a polytopic membrane protein, (vii) a pump protein, (viii) a channel protein, (iX) a receptor kinase protein, (X) a G protein-coupled receptor protein, Xii) a membrane-associated enzyme, and (Xiii) a transporter protein, wherein the hydrophobic protein is bound by amphiphile selected from the group consisting of (i) a polar lipid, (ii) an amphiphilic macromolecular polymer, (iii) a surfactant or detergent, and (iV) an amphiphilic polypeptide; (b) selecting a ligand molecule using multi-dimensional chromatography by affinity selection by exposing under homogenous or heterogeneous solution phase conditions the hydrophobic target protein bound by an amphiphile to a multiplicity of molecules from a mass-coded library to promote the formation of at least one complex between the hydrophobic target protein and the ligand molecule, (c) separating the complex from the unbound molecules, and (d) identifying the ligand molecule by mass spectral analysis.

In a fifth aspect, the invention provides a method for identifying a ligand for a hydrophobic protein, the method comprising (a) selecting a hydrophobic target protein from the group consisting of (i) a membrane protein, (ii) an integral membrane protein, (iii) a transmembrane protein, (iv) a monotopic membrane protein, (v) a polytopic membrane protein, (vii) a pump protein, (viii) a channel protein, (iX) a receptor kinase protein, (X) a G protein-coupled receptor protein, Xii) a membrane-associated enzyme, and (Xiii) a transporter protein, wherein the hydrophobic protein is bound by amphiphile selected from the group consisting of (i) a polar lipid, (ii) an amphiphilic macromolecular polymer, (iii) a surfactant or detergent, and (iV) an amphiphilic polypeptide; (b) selecting a ligand molecule using multi-dimensional chromatography by affinity selection by exposing under homogenous or heterogeneous solution phase conditions the hydrophobic target protein bound by an amphiphile to a multiplicity of molecules from a library that is not mass-coded to promote the formation of at least one complex between the hydrophobic target protein and the ligand molecule, (c) separating the complex from the unbound molecules, and (d) identifying the ligand molecule by mass spectral analysis.

In a sixth aspect, the invention provides a method of isolating a hydrophobic protein, the method comprising: (a) selecting a hydrophobic protein comprising: (i) at least one transmembrane domain sequence, (ii) at least two tag sequences useful for affinity selection selected from the group consisting of: (A) a FLAG tag (NH2-DYKDDDDK-COOH) (SEQ ID NO:29), (B) an EE tag (NH2-EEEEYMPME-COOH) (SEQ ID NO:30), (C) a hemagglutinin tag (NH2-YPYDVPDYA-COOH) (SEQ ID NO:31), (D) a myc tag (NH2-KHKLEQLRNSGA-COOH) (SEQ ID NO:32), and (E) an HSV tag (NH2-QPELAPEDPED-COOH) (SEQ ID NO:33); (iii) a hydrophobic protein (HP) sequence selected from the group consisting of: (A) a membrane protein, (B) an integral membrane protein, (C) a transmembrane protein, (D) a monotopic membrane protein, (E) a polytopic membrane protein, (F) a pump protein, (G) a channel protein, (H) a receptor kinase protein, (I) a G protein-coupled receptor protein, (J) a membrane-associated enzyme, and (K) a transporter protein; (b) purifying the hydrophobic protein by sucrose gradient ultracentrifugation; (c) purifying the hydrophobic protein by antibody affinity purification; and (d) purifying the hydrophobic protein by immobilized metal affinity chromatography.

In a seventh aspect, the invention provides, an isolated nucleic acid molecule suitable for hydrophobic protein expression, comprising: (a) a vector polynucleotide sequence for protein expression in a eukaryotic cell, and (b) a polynucleotide sequence encoding an engineered hydrophobic protein comprising the following elements (i) an N-terminal methionine residue, (ii) a heterologous signal sequence (SS), wherein the N-terminal methionine sequence and the heterologous signal sequence are selected from the group consisting of (1) MKFLVNVALVFMVVYISYIYA (SEQ ID NO:24), (2) MVRTAVLILLLVRFSEP (SEQ ID NO:25), (3) MKTIIALSYIFCLVFA (SEQ ID NO:26), (4) MMNGTEGPNFYVPFSNKTGVVRSPFEAPQYYLAEP-COOH (SEQ ID NO:27) and (5) MGKNPLGVRKTETSQVAPA-COOH (SEQ ID NO:28); (iii) at least one transmembrane domain sequence, (iv) at least two tag sequences useful for affinity selection selected from the group consisting of (1) a FLAG tag (NH2-DYKDDDDK-COOH) (SEQ ID NO:l), (2) an EE tag (NH2-EEEEYMPME-COOH) (SEQ ID NO:2), (3) a hemagglutinin tag (NH2-YPYDVPDYA-COOH) (SEQ ID NO:3), (4) a myc tag (NH2-KHKLEQLRNSGA-COOH) (SEQ ID NO:4), and (5) an HSV tag (NH2-QPELAPEDPED-COOH) (SEQ ID NO:5), and (v) a hydrophobic protein (HP) sequence selected from the group consisting of (1) a membrane protein, (2) an integral membrane protein, (3) a transmembrane protein, (4) a monotopic membrane protein, (5) a polytopic membrane protein, (6) a pump protein, (7) a channel protein, (8) a receptor kinase protein, (9) a G protein-coupled receptor protein, (10) a membrane-associated enzyme, and (11) a transporter protein.

The following examples are intended to further illustrate certain preferred preferred embodiments of the invention but are not meant to limit the scope of the invention in any way.

### EXAMPLES

### EXAMPLE 1

### Affinity Selection of COX-1 Ligands and Identification by Alis

Purified ovine COX-1 (>95% by SDS-PAGE) from Cayman Chemical Company (Ann Arbor, Mich.) was prepared for screening by exchanging the detergent. To remove the detergent in which the protein was supplied, Tween-20, ion exchange chromatography was conducted. Approximately 6 mL of 0.27 mg/mL COX-1 in a buffer of 80 mM Tris-8.0, 0.09% Tween-20, 270 μM DDC, and 240 μM dodecyl-β-D-maltoside (DβM), which would provide a theoretical yield of 1.8 mg, was applied to a Poros HQ column with a buffer of 80 mM tris, pH 8.0, 240 μM DβM (TBS-AGD). The column was developed with a linear gradient from 0 to 0.5 M NaCl over 10 minutes with a flow rate of 5 mL/minute. The eluted protein fractions were identified by monitoring absorbance at 280 nm.

After this treatment, 18 mL of protein-containing material were pooled and concentrated in an Amicon-30 centricon according to the manufacturer's instructions (Millipore, Inc.; Bedford, Mass.). This yielded a concentration of approximately 1.8 mg/ml of COX-1 which was diluted to 1.3 mg/mL (20 μM COX-1) for screening. It is estimated that the buffer in the final protein preparation consisted of 2.4 mM DβM, 80 mM tris-8.0, about 50 mM NaCl. This COX-1 solution was promptly supplemented with 20 μM hemin, and 300 μM diethyldithiocarbamate (DDC) in accordance with the handling procedures used by Cayman Chemical company.

The COX-1 protein preparation was then used for sample preparation according to the appended sample preparation standard operating procedure (SOP, Table 2).

TABLE 2
Hydrophobic Protein Ligand Screening Procedure

| Binding Mixture Components: | | |
|---|---|---|
| Final Protein Concentration | | 10 μM |
| Final library concentration | | 2.5 mM |
| Final discrete ligand concentration | | 1 μM |
| Total volume (for single injection) | | 12 μL |
| Stock Reagents: | | |
| COX-1 Target Protein (2X, stored at −80 degrees) | | 1.3 mg/mL (20 μM) in 80 mM Tris/240 μM DBM |
| BlacB Control Protein (6X, stored at 4 degrees) | | 5.5 mg/mL (˜150 μM) BlacB in 1X SEC |
| Library (40X, stored at 80 degrees) | | 100 mM in DMSO |
| Discrete Ligands (40X, stored at −80 degrees) | | 40 μM in DMSO |
| SEC Premix: | | |
| SEC Buffer Stock (1M Tris 8.0) | | 400 μL |
| Water | | 4.6 μL |
| Screening Procedure | | |
| 1. | Prepare SEC premix solution stock (80 mM Tris 8.0) | |
| | and pre-equilibrate to 42 degrees Celsius | |
| 2. | Check that stocks of libraries/ligands in DMSO are | |
| | prepared and thawed to room temperature. | |
| 3. | Add labels from database to each 250 μL polypropylene | |
| | autosampler vial | |
| 4. | Add each library/ligand stock to a 500 μL siliconized | 1 μL of a 100 mM library stock or |
| | tube; store overnight at −80 degrees C. | 1 μL of a 40 μM discrete ligand stock |
| 5. | For protein controls w/o library/ligand, add DMSO | 1 μL DMSO |
| | instead of ligand/library stock | |
| 6. | Thaw pre-aliquoted library/ligand stocks at room | 19 μL warm premix |
| | temp.; add warm premix to each library and mix | |
| | immediately by pipetting and vortexing | |
| 7. | Centrifuge at maximum RPM at room temp. for at | |
| | least 10 minutes | |
| 8. | Thaw target and control protein stocks on ice | |
| | Give to Analytical to exchange detergent to DBM | |
| | (3 ml (500K units) is enough for 50-75 samples) | |
| 9. | Concentrate dilute COX-1, ˜10x to 1.3-1.5 mg/ml with | |
| | Centricon-30's. Determine protein conc. With Bio-Rad | |
| | assay using BSA 1 mg/ml (Sigma P 0914) as reference | |
| | standard. Assay centricon flowthrough for protein and | |
| | correct [COX-1] | |
| 10. | To COX-1, add 1 equivalent hemin (Sigma 3281, | |
| | 3.3 μM in DMSO) and DDC (Sigma D3056) to 300 μM | |
| | keep protein on ice until needed. | |
| 11. | Carefully transfer clarified supernatants to new poly- | 6 μL supernatant |
| | propylene autosampler vials | |
| 12. | In cold room, aliquot target or control protein stock to vials | 6 μL protein |
| | containing libraries and mix well by pipetting | |

Incubate protein + library/ligand mixtures for 30 minutes
at 4 degrees (no RT incubation)
Notes:
For SEC chromatography: 50 mm GPC-Peptide columns
run in 50 mM Tris pH 8.0, 150 mM NaCl, 2.5% DMSO

This sample prep SOP yields binding assays that combine COX-1 with mass-encoded combinatorial libraries made of approximately 2500 small drug-like molecules, each member at a concentration of 1 μM. The sample was incubated 30-minutes at 4° C. As the total volume was 12 μL, the sample thus contained 240 pmol protein and 12 pmol of each library component. As a control test, an unrelated membrane protein, diacyl glycerol kinase (Calbiochem, Inc.; San Diego, Calif.), was also prepared in the same buffer at 20 μM and incubated with the same 2500-member library and treated similarly.

Then the mixtures were individually subjected to ALIS Analysis. If any ligand of suitably high affinity was bound to the COX at the time its fraction was collected, the mass spectral analysis would identify its mass. By virtue of the mass-coding, the precise combination of building blocks and core molecule can be identified (see U.S. Pat. No. 6,147,344). If the same compound failed to appear in the diacyl glycerol kinase control experiment, the compound may then be identified as a specific ligand of the COX-1.

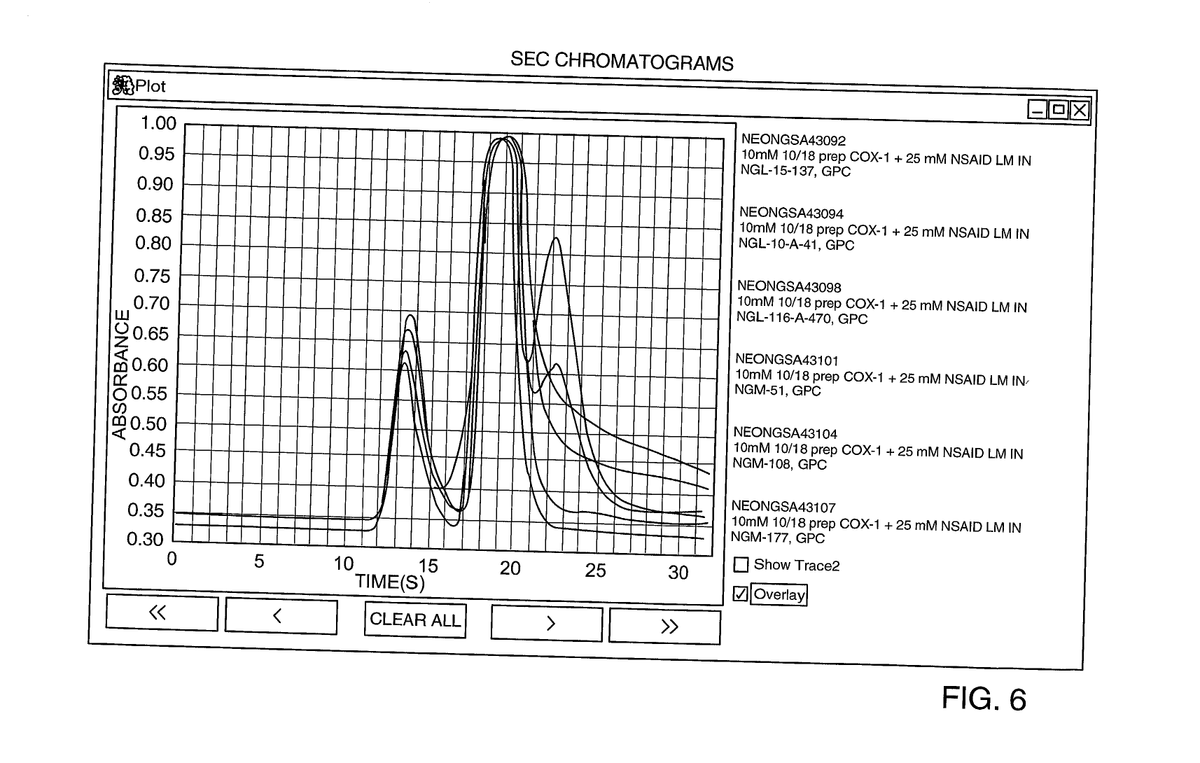

The mixtures were then individually subjected to modified ALIS analysis as follows. The large detergent-solubilized protein was separated from the small drug-like molecules by size exclusion chromatography (SEC) over a 4.6 mm×50 mm×5 μm SEC column at 0° C. using a running buffer of TBS (80 mM tris, pH 8.0, 150 mM NaCl, 2.5% DMSO) at a flowrate of 2 mL/minute. The eluting SEC fraction-containing protein was identified by UV-VIS detection monitoring at 230 nm and transferred by way of a sample loop to a low-flow (100 μL/minute) reverse-phase chromatography (RPC) system. The RPC column (Higgins C-18; 1 mm×50 mm×5 μm) is maintained at 60° C. to promote dissociation of ligands from the complex. From this RPC column, the ligand is eluted into a high-resolution mass spectrometer for analysis using a gradient of 5%-95% acetonitrile (0.1% formic acid counterion) in water (w/0.1% formic acid) over 5 minute. If any ligand of suitably high affinity was bound to the COX at the time its fraction was collected, the mass spectral analysis will identify its mass. By virtue of the mass encoding the precise combination of building blocks and core can be identified (see U.S. Pat. No. 6,147,344 by Annis et al.). If the same compound failed to appear in the diacyl glycerol kinase control experiment, the compound would be identified as a specific ligand of the COX-1. FIG. 6 illustrates the separation of protein from unbound small molecules using ALIS.

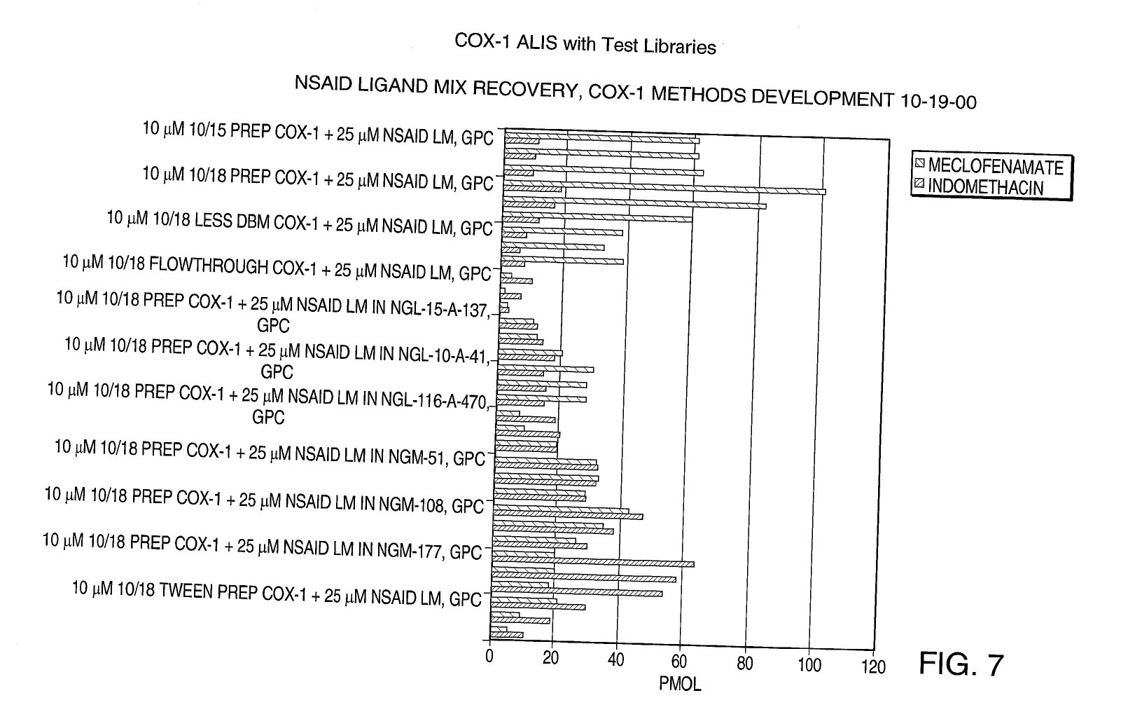

Control experiments demonstrated that COX-1 screened in this manner enabled known COX-1 ligands to be extracted from large mixtures of small molecules (FIG. 7). When a test library composed of 25 μM meclofenamate, 25 μM indomethacin, 1 μM each of various test libraries, these known COX-1 ligands are recovered and identified by the ALIS screening method.

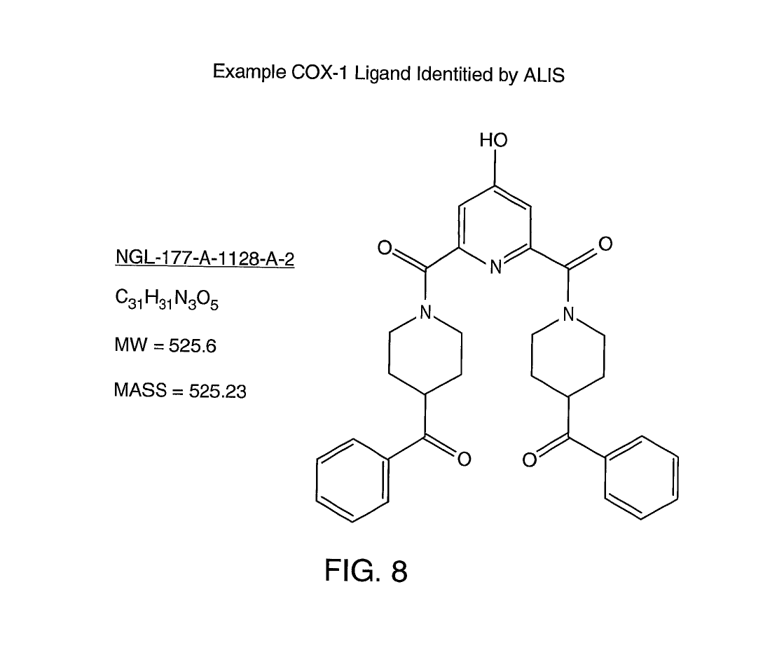

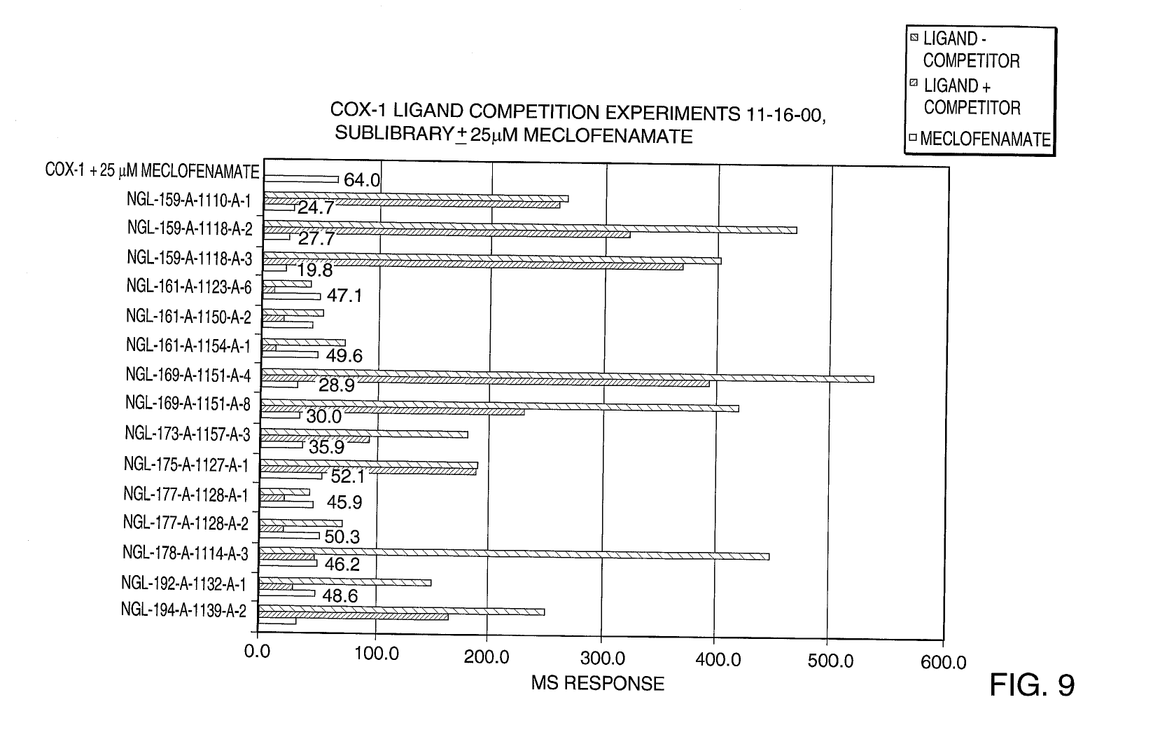

This experiment demonstrated that after screening over 330,000 small drug-like molecules, 41 small molecules were identified as COX-1 ligands, one example of which is shown in FIG. 8. These COX-1 ligand molecules were identified in two screens against small libraries, and their single molecule formulations are in preparation for further testing. Furthermore, many of these hits are observed to compete with known COX-1 ligand, meclofenamate, for binding (FIG. 9).

### EXAMPLE 2

### Identification of Ligand Binding to m2 mAChR Protein by Mass Spectroscopy

A gene construct encoding the m2 subtype of the muscarinic acetylcholine receptor (m2R) was cloned into a baculovirus expression vector according to conventional cloning methods (see e.g., Baculovirus Expression Vector System, 6th Edition, 1999, Pharmingen, San Diego, Calif.). The gene construct encoded a polypeptide with an amino terminal methionine followed immediately in frame by the melittin signal sequence (SEQ ID NO:12) followed immediately in frame by the FLAG M1 epitope tag (SEQ ID NO:1) followed immediately in frame by the sequence for the m2 muscarinic acetylcholine receptor (NCBI Accession No. X04708). The full-length polypeptide sequence therefore was:

| | |
|---|---|
| NH2- | |
| MKFLVNVALVFMVVYISYIYADYKDDDDKMMNNSTNSSNSGLALTS | (SEQ ID NO:34) |
| PYKTFEVVFIVLVAGSLSLVTIIGNILVMVSIKVNRHLQTVNNYFL | |
| FSLACADLIIGVFSMNLYTLYTVIGYWPLGPVVCDLWLALDYVVSN | |
| ASVMNLLIISFDRYFCVTKPLTYPVKRTTKMAGMMIAAAWVLSFIL | |
| WAPAILFWQFIVGVRTVEDGECYIQFFSNAAVTFGTAIAAFYLPVI | |
| IMTVLYWHISRASKSRIKKDKKEPVANQEPVSPSLVQGRIVKPNNN | |
| NMPGSDEALEHNKIQNGKAPRDAVTENCVQGEEKESSNDSTSVSAV | |
| ASNMRDDEITQDENTVSTSLGHSKDENSKQTCIKIVTKTQKSDSCT | |
| PANTTVELVGSSGQNGDEKQNIVARKIVKMTKQPAKKKPPPSREKK | |
| VTRTILAILLAFIITWAPYNVMVLINTFCAPCIPNTVWTIGYWLCY | |
| INSTINPACYALCNATFKKTFKHLLMCHYKNIGATR-COOH | |

Upon expression, the mellitin signal sequence is cleaved after Ala(21) revealing an amino terminal FLAG epitope which is bound specifically by the FLAG M1 antibody resin (Sigma; St. Louis, Mo.). This baculovirus expression vector was used to generate baculovirus that directed the expression of the above polypeptide in insect cells according the conventional methods (Baculovirus Expression Vector System, 6th Edition, 1999, Pharmingen, San Diego, Calif.).

To purify FLAG-tagged m2R, 60 g of insect cells expressing the above polypeptide were suspended in 0.6 L of TBS [50 mM Tris-CL, pH 7.4, 100 mM NaCl), and the sample was homogenized by nitrogen cavitation. The homogenate was subjected to centrifugation at 500×g for 30 minutes at 4° C. to removed non-homogenized cells. The pellet was discarded and the supernatant was subjected to ultracentrifugation at 100,000×g for 45 minutes at 4° C. The ultracentrifugation supernatant was discarded and the pelleted cell membranes were resuspended in TBS containing 0.5% (w/v) digitonin (TBS-D buffer) to a protein concentration of 2.5 mg/mL. This suspension was incubated, stirring for 60 minutes at 4° C. before ultracentrifugation at 100,000×g for 45 minutes at 4° C. to pellet insoluble material. The soluble supernatant was applied to a 5 mL column of the FLAG M1 antibody resin pre-equilibrated with TBS-D buffer at a flow rate of 0.7 mL/min for antibody affinity purification. After loading the soluble material onto the FLAG M1 antibody resin, the column was washed with 50 mL of TBS-D buffer and then the FLAG-tagged m2R protein was eluted from the column with TBS-D buffer containing 100 :g/mL of FLAG peptide (Sigma; St. Louis, Mo.). Eluted column fractions containing purified FLAG-tagged m2R were identified by SDS-PAGE.

To assess the concentration of purified FLAG-tagged m2R protein that was capable of binding muscarinic ligands, glass fiber filter-binding assays were performed according to the method of Peterson (Peterson, G. L., et al (1995) J. Biol. Chem. 270:17808).

This m2R preparation consisted of 6 :M m2R in TBS-D with 100 :g/mL of FLAG peptide. Each m2R polypeptide is reversibly associated with a multiplicity of digitonin molecules, creating a membrane protein-detergent complex with a stoichiometry of m2R:digitonin of 1:5-500. This preparation was designated as Stock m2R for use in ALIS sample preparation according to the sample preparation protocol outlined below.

Other stock reagents were prepared. Stock cyclooxygenase 1 (COX) was prepared according to the method in Example 1 to yield a concentration of 6 :M COX in TBS. Stock discrete ligands (Sigma; St.Louis, Mo.) pirenzepine, quinuclidinyl benzylate (QNB), and atropine were prepared to 400 :M in TBS. Four combinatorial chemical libraries were prepared in dimethyl sulphoxide (DMSO) constituting on average 2500-5000 small drug-like molecules each at a concentration of 400 :M. These four drug libraries were designated NMG-66, NGM-41, NGL-10-A-41, and NGL-116-A-470. Four stock test libraries were prepared containing 400 :M atropine by adding atropine dissolved in DMSO to the individual drug libraries mentioned above to yield Stock NMG-66 Plus Atropine, Stock NGM-41 Plus Atropine, Stock NGL-10-A-41 Plus Atropine, and Stock NGL-116-A-470 Plus Atropine. Four stock test libraries were prepared containing 400 :M QNB by adding QNB dissolved in DMSO to the individual drug libraries mentioned above to yield Stock NMG-66 Plus QNB, Stock NGM-41 Plus QNB, Stock NGL-10-A-41 Plus QNB, and Stock NGL-116-A-470 Plus QNB. Premix Buffer was prepared by combining 100 :L of 5% digitonin, with 4.6 mL water, and 400 :L 1 M Tris-Cl, pH 7.5, then equilibrated at 42° C.

Binding reactions were prepared that combined protein (either m2R test protein or COX control protein) with ligand (either QNB alone, atropine alone, pirenzepine alone, atropine plus drug library, or QNB plus drug library) or protein with DMSO as a control. In each case, 38 :L of Premix Buffer was dispensed into polypropylene tubes containing 2 :L of DMSO or DMSO-solubilized ligands, mixed by vortexing, and centrifuged at 8,000×g for 10 minutes at room temperature to remove insoluble material. The clarified supernatants (2 :L) containing either aqueous DMSO alone or aqueous DMSO-solubilized ligands (with or without drug libraries) were transferred to polypropylene tubes at 4° C. Target protein (8 :L) m2R or control (8 :L) protein COX was added to the supernatants, mixed well by pipetting, and incubated at 4° C. for 60 minutes.

These binding reaction preparations were then subjected to ALIS analysis. The binding reaction preparations combine 4.8 :M target membrane protein (m2R) or 4.8 :M control membrane protein (COX) with a multiplicity of approximately 2500 small drug like molecules each at a concentration of 1-10 :M in a manner that established equilibrium binding conditions. High affinity ligands (K_{id}<100 :M) to the proteins are then identified by ALIS. Small molecule ligands of the target protein m2R that do not also bind to the control protein COX are considered as specific ligands of the target protein. Separately, for comparison to experiments with a multiplicity of drug molecules, binding reactions were also prepared combining m2R or COX with individual (discrete) m2R ligands. This ALIS Analysis proceeded as described below with a series of size exclusion chromatography (SEC), followed by reverse phase chromatography (RPC), followed by mass spectrometric (MS) analysis.

These prepared binding reaction mixtures were individually subjected to ALIS Analysis. Ligands that bound to the m2R with suitably high affinity were collected with the protein-containing SEC fraction as follows. The large detergent-solubilized molecules were separated from the unbound small drug-like molecules by SEC over a 4.6 mm×50 mm×5 :m SEC column at 4° C. using a running buffer of 50 mM Tris-Cl, pH 7.5, 150 mM NaCl, 2.5% DMSO at a flowrate of 2 mL/minute. The protein containing fraction was identified with ultraviolet electronic absorption spectrometry monitoring at 230 nm.

The protein-containing fraction was transferred by way of a sample loop to a low flowrate (100 :L/min RPC system. The RPC column (Higgins C-18; 1 mm×50 m×5 :m) is maintained at 60° C. to promote ligand dissociation from the complex. From this RPC column the ligand in eluted into a high-resolution mass spectrometer for analysis using a gradient of 5%-95% acetonitrile (0.1% formic acid counterion) in water (w/0.1% formic acid) over 5 minutes. Mass analyzed ligands collected with the protein-containing SEC fraction by virtue of its high affinity for the protein-detergent m2R complex were identified by fore-knowledge of their precise mass.

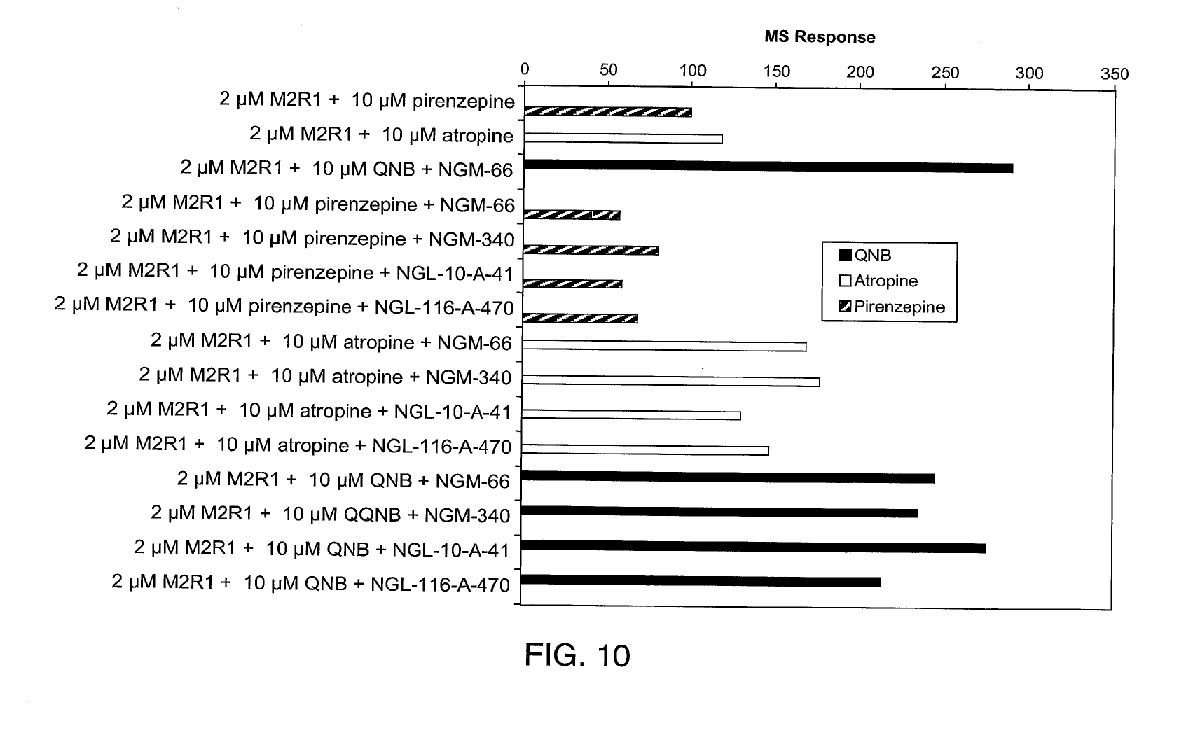

Experiments demonstrated that m2R screened by ALIS Analysis in this manner enabled known m2R ligands to be extracted from mixtures of a multiplicity of small drug like molecules by virtue of the known m2R ligands' high affinity for the m2R-detergent complex. This ALIS-formatted screen recovered m2R ligands from drug libraries just as well as it recovered m2R ligands bound to the m2R-detergent complex in the absence of drug libraries (FIG. 10).

Data for these experiments are presented in FIG. 10. In these experiments, equilibrium binding reactions were established by combining 2.0 :M m2R with 10 :M pirenzepine, QNB, or atropine in the absence or presence of combinatorial drug libraries (NGM-66, NGM-340, NGL-10-A-41, NGL-116-A-470) that presented approximately 2500 small drug-like molecules each at a concentration of 1-10 :M. The binding reactions were subjected to ALIS Analysis and the extent of ligand recovery was quantified by the signal strength of the mass spectrometer. The x-axis is in relative units of mass spectrometric signal response for the respective masses of pirenzepine, QNB, and atropine.

### EXAMPLE 3

### Displacement Assay in an Affinity-Based Selection and Alis Identification of HP Ligands

As a representative HP, m2 mAChR is purified according to the method of Peterson et al. (Peterson, G. L. et al. (1995) J. Biol. Chem. 270: 17808). The target protein is adjusted to a concentration of 20 :M in a buffer of TBS-AGD and is incubated with a known muscarinic ligand, pirenzepine (MW=424.3), which is at a concentration of 1 :M. As a control test, an unrelated membrane protein, glycophorin, is also prepared in TBS-AGD at 20 :M and is incubated with the compound pirenzepine (MW=424.3), which is at a concentration of 1 :M.

A library of mass-coded compounds is added to the m2 mAChR/pirenzepine mixture, and the sample is analyzed to determine if a library compound displaces pirenzepine from the HP protein. Displacement of pirenzepine indicates that the library compound binds more tightly and at the same site as pirenzepine itself.

The displacement assay is conducted as follows. The mixture of m2 mAChR, 1 :m pirenzepine, and 1 :M of each library compound are subjected to ALIS Analysis. In this analysis MS signal corresponding to the mass of pirenzepine is monitored, while the mass of the library compounds are ignored. If the library compound quantitatively reduces the MS signal corresponding to the mass of pirenzepine, it is inferred that the library compound displaced pirenzepine from the HP protein, in this case mAChR protein. If incubation with the library compounds does not alter the MS signal corresponding to the mass of pirenzepine, the library compounds are considered not to contain an m2 mAChR ligand. By comparing pirenzepine MS signal with and without incubation with the library compounds, one can assess whether the library compound displaces pirenzepine and thus binds to the mAChR protein.

### EXAMPLE 4

### Affinity Selection of m2 mACHR Mass-Coded Ligands and Identification by Alis

As a representative HP, m2 mAChR is purified according to the method of Peterson et al. (Peterson, G. L. (1995) J. Biol. Chem 270: 17808). The protein is adjusted to 20 :M in a buffer of TBS-AGD is incubated with a 2500-member library of mass-coded compounds, each member at a concentration of 1 :M. After a 30 minute incubation at 22° C., the sample was chilled at 4° C. pending ALIS analysis. As a control test, an unrelated membrane protein, glycophorin, is also prepared in TBS-AGD at 20 :M and incubated with the mass-coded library. To determine if the compound specifically binds to the mAChR, the mAChR-compound mixture is analyzed by ALIS Analysis. If a mass corresponding to one of the members of the mass-coded library appears when the protein peak is collected and surveyed by MS, that compound may be identified as a binding ligand. If the same compound fails to appear in the glycophorin control experiment, the compound may then be identified as a specific ligand of the mAChR. By virtue of the mass encoding the precise combination of building blocks and core can be identified (see U.S. Pat. No. 6,147,344 by Annis et al.). Using MS-MS analysis (see U.S. Pat. No. 6,147,344 by Annis et al.), the exact structure of the core plus building block combination can also be pinpointed.

### EXAMPLE 5

### Affinity Selection of COX-1 Ligands and Identification by Modified Alis with On-Line Fluorescence Detection

As a representative HP, COX-1 protein was purified from ram seminal vesicles according to the method of Johnson et al. (Johnson, J. L. (1995) Arch. Biochem. Biophys. 324:26-34). The COX-1 sample is adjusted to 20 :M in TBS-AGD (50 mM tris, pH 8.0, 150 mM NaCl, 800 mM dodecyl ∃-D-maltoside, 2.5% DMSO) is mixed and incubated with a 2500-member library of mass-coded compounds, each member at a concentration of 1 :M. After a 30 minute incubation at 22° C., the sample was chilled at 4° C. pending ALIS analysis. As the total volume is 12 :L, the sample thus contains 240 pmol protein and 12 pmol of each library component. As a control test, an unrelated membrane protein, glycophorin, is also prepared in TBS-AGD at 20 :M and incubated with the same 2500-member library and treated similarly.

Then the mixtures are individually subjected to modified ALIS analysis as follows. The large detergent-solubilized protein is separated from the small drug-like molecules by size exclusion chromatography (SEC) over a 4.6 mm×50 mm×5 :m SEC column at 0° C. using a running buffer of TBS (50 mM tris, pH 8.0, 150 mM NaCl, 2.5% DMSO) at a flowrate of 2 mL/minutes. The eluting SEC fraction containing protein is identified by on-line fluorescence detection exciting at 240-250 nm and monitoring emission at 340 nm and transferred by way of a sample loop to a low-flow (100 :L/minute) reverse-phase chromatography (RPC) system. The RPC column (Higgins C-18; 1 mm×50 mm×5 :m) is maintained at 60° C. to promote dissociation of ligands from the complex. From the RPC column, the ligand is eluted into a high-resolution mass spectrometer for analysis using a gradient of 5%-95% acetonitrile (0.1% formic acid counterion) in water (w/0.1% formic acid) over 5 minutes. Ligand of suitably high affinity bound to the COX-1 at the time its fraction is collected, and the mass of the ligand is identified by mass spectral analysis.

Through the mass coding, the precise combination of building blocks and core molecule are identified (see U.S. Pat. No. 6,147,344 by Annis et al.). If the same compound fails to appear in the glycophorin control experiment, the compound may then be identified as a specific ligand of the COX-1 protein.

### EXAMPLE 6

### Affinity Selection of COX-1 Ligands and Identification by Modified Alis with On-Line Light Scattering Detection

As a representative HP, COX-1 protein was purified from ram seminal vesicles according to the method of Johnson et al. (Johnson, J. L. (1995) Arch. Biochem. Biophys. 324:26-34). The COX-1 sample is adjusted to 20 :M in TBS-AGD (50 mM tris, pH 8.0, 150 mM NaCl, 800 mM dodecyl ∃-D-maltoside, 2.5% DMSO) is mixed and incubated with a 2500-member library of mass-coded compounds, each member at a concentration of 1 :M. After a 30 minute incubation at 22° C., the sample is chilled at 4° C. pending modified ALIS analysis. As the total volume is 12 :L, the sample contains 240 pmol protein and 12 pmol of each library component. As a control test, an unrelated membrane protein, glycophorin, is also prepared in TBS-AGD at 20 :M and incubated with the same 2500-member library and treated similarly.

Next, the control and test mixtures are individually subjected to modified ALIS analysis as follows. The large detergent-solubilized protein is separated from the small drug-like molecules by size exclusion chromatography (SEC) over a 4.6 mm×50 mm×5 :m SEC column at 0° C. using a running buffer of TBS (50 mM tris, pH 8.0, 150 mM NaCl, 2.5% DMSO) at a flowrate of 2 mL/minute. The eluting SEC fraction containing protein is identified by on-line light scattering detection and transferred by way of a sample loop to a low-flow (100 :L/minute) reverse-phase chromatography (RPC) system. The RPC column (Higgins C-18; 1 mm×50 mm×5 :m) is maintained at 60° C. to promote dissociation of ligands from the complex. From the RPC column, the ligand is eluted into a high-resolution mass spectrometer for analysis using a gradient of 5%-95% acetonitrile (0.1% formic acid counterion) in water (w/0.1% formic acid) over 5 minutes. Ligand of suitably high affinity bound to the COX-1 protein at the time its fraction is collected, and the mass of the ligand is identified by mass spectral analysis.

Through the mass coding, the precise combination of building blocks and core molecule are identified (see U.S. Pat. No. 6,147,344 by Annis et al.). If the same compound fails to appear in the glycophorin control experiment, the compound may then be identified as a specific ligand of the COX-1 protein.

### EXAMPLE 7

### Heterogeneous Solution Phase Screening for Ligands that Bind m2 mACHR: Identification Using Affinity Selection and Alis Analysis

HP ligands may also be screened by utilizing a heterogeneous solution phase screening method in which a tagged target sequence is immobilized on a solid support. For example, anti-flag antibody-loaded protein A agarose beads (anti-flag beads) are prepared for use as a sedimentable stationary element in an immunoprecipitation (IP)-based screening protocol.

Briefly, using a buffer of TBS-AG (50 mM tris, pH 8.0, 150 mM NaCl, 800 mM dodecyl ∃-D-maltoside), 100 :L of 50% v/v slurry of protein A agarose (Santa Cruz Biotechnology, St. Louis, Mo.) are washed in a 1.5 mL eppendorf tube with three room temperature cycles of: (1) combining the beads with 1 mL TBS-AG; (2) mixing the sample by tumbling for 20 minutes; and (3) centrifugation at 10000×g, followed by a careful removal of the supernatant that leaves the pelleted agarose beads in the bottom of the tube.

After the beads have been washed, the 50 :L pellet of beads is brought up in 1.0 mL TBS-AG. To that mixture, 50 :L of 1 mg/mL ∃-galactosidase in TBS-AG buffer is added and the mixture is incubated at 4° C. for 60 minutes to block non-specific binding of protein to the beads. To that mixture is added 10 :L of 1.0 :g/mL anti-flag antibody (Sigma, St. Louis, Mont.), and the mixture is incubated at 4° C. for 60 minutes. In parallel, another preparation of control agarose beads handled similarly is treated with 10 :L of TBS-AG instead of the anti-flag antibody. The anti-flag loaded beads and the control beads are then washed to remove excess antibody and protein and, finally, resuspended in 0.10 mL of TBS-AG buffer and transferred to 0.5 mL eppendorf tubes.

CHO cells expressing an m2 mAChR-flag tag-His tag protein are cultured, lysed, and homogenized. The m2 mAChR-containing membranes of the cells are purified by sucrose step-gradient ultracentrifugation. This sucrose step-gradient ultracentrifugation step removes most non-membrane proteins and cell debris. The ultracentrifugation step also has the potential of isolating specific populations of membranous cellular substructures. The mAChR-enriched membranes are solubilized with detergent (dodecyl-∃-maltoside, D∃M or CYMAL-7; (Anatrace; Maumee, Ohio)) and subjected to three steps of affinity purification.

First, metal chelate affinity chromatography (MCAQ will be used to take advantage of the polyhistidine-tagged tagged C-terminus, followed by a second affinity purification, an antibody affinity purification based on the FLAG-tagged N-terminus (Kobilka, B. K. (1995) Anal. Biochem. 231: 269). Third, ligand affinity purification over a column of immobilized mAChR ligand 3-(2′-aminobenzhydryloxy)-tropane (ABT), followed by a desalting step will yield a final enrichment of active m2 mAChR protein.

The sample is adjusted to 20 :M m2 mAChR protein in a buffer of TBS-AGD and is incubated with a 2500-member library of mass-coded compounds, each member at a concentration of 1 :M. The reaction is done in a volume of 40 :L. After a 30 minute incubation at 22° C., the sample is chilled at 4° C. pending MS analysis. As a comparison test, ∃-galactosidase is similarly prepared in TBS-AGD at 20 :M and incubated with the mass-coded library.

The m2 mAChR protein-compound mixtures are prepared for analysis by MS analysis with the following procedure. The purified protein-library mixtures, either the m2 mAChR protein or ∃-galactosidase protein trials, are each split into two 20 :L volumes. For the m2 mAChR-library mixture, one volume is combined with the anti-flag beads (anti-flag/protein A agarose) for IP and the other volume is subjected to a mock IP by combination with the control agarose beads (buffer/protein A agarose beads). Similarly, for the ∃-galactosidase mixture, one 20 :L volume is combined with the anti-flag beads for a control IP and the other 20 :L volume is subjected to a mock IP by combination with the control agarose beads. These IPs proceed, mixed by tumbling, for 60 minutes at 4° C. Afterwards, each IP is washed with three room temperature cycles of: (1) combining the beads with 1 mL TBS-AG, (2) mixing by tumbling for 2 minutes, and (3) centrifugation at 10,000 g, followed by a careful removal of the supernatant that leaves the pelleted agarose beads in the bottom of the tube. Finally, each 50 :L bed volume bead preparation is then resuspended in an additional 50 :L of TBS (50 mM tris, pH 8.0, 150 mM NaCl) and kept at 4° C. At the completion of this process, four heterogeneous bead preparations are made: (1) m2 mAChR/anti-flag/protein A agarose (m2 beads), (2) m2 mAChR/buffer/protein A agarose beads (m2 control beads), (3) ∃-galactosidase/anti-flag/protein A agarose beads (∃-lac beads), and (4) ∃-galactosidase/buffer/protein A agarose beads (∃-galactosidase control beads).

These bead-based preparations constitute heterogeneous solution phase systems useful for screening in the following manner. Each 100 :L bead preparation is combined with 50 μL of 30% acetonitrile in water heated to 60° C. for 5 minutes to dissociate bound ligands from the protein. Then the mixture is centrifuged at 10,000×g to pellet the beads, and the dissociated protein in the supernatant is transferred to a new tube.

The IP procedure allows both an affinity selection of a ligand and the physical separation of the m2mAChR-ligand complex from unbound library members. These supernatants contain 50 mM tris, pH 8.0, 150 mM NaCl, 80 mM dodecyl ∃-D-maltoside, and 10% acetonitrile. Approximately 60 :L of these supernatant samples are injected individually into a low-flow (100 :L/minutes) reverse-phase chromatography (RPC) system. The RPC column (Higgins C-18; 1 mm×50 mm×5 :m) is maintained at 60° C. From this RPC column, the ligand is eluted into a high-resolution mass spectrometer for analysis using a gradient of 5%-95% acetonitrile (0.1% formic acid counterion) in water (w/0.1% formic acid) over 5 minutes.

m2 mAChR-ligands are identified by observing a mass corresponding to one compounds from the 2500-member mass-coded library in the supernatant of the m2mAChR protein beads and not from the supernatant of the m2mAChR protein control, ∃-lac library, or ∃-lac control beads. The structure of the mass-coded compounds selected in the procedure are easily identified (see U.S. Pat. No. 6,147,344 by Annis et al.). Using MS-MS analysis (see U.S. Pat. No. 6,147,344 by Annis et al.), the exact structure of the core plus building block combination can also be pinpointed.

### EXAMPLE 9

### Heterogeneous Solution Phase Screening for Ligands that Bind m2 mACHR: Identification Using a Displacement Assay Combined with Affinity Selection and MS Analysis

Flag-tagged m2 mAChR protein is purified as outlined herein above and resuspended at a concentration of 20 :M in a buffer of TBS-AGD. The protein is incubated with a 2500-member library of mass-coded compounds, each member at a concentration of 1 :M and with 1 :M pirezepine, a known ligand for m2 mAChR protein, in a volume of 40 :L. After a 30 minute incubation at 22° C., the sample was chilled at 4° C. pending MS analysis. As a comparison test, ∃-galactosidase is similarly prepared in TBS-AGD at 20 :M and incubated with the mass-coded library and pirezepine.

The purified protein-library-pirenzepine mixtures, from either the m2 mAChR protein or ∃-galactosidase trials, are each split into two 20 :L volumes. For the m2 mAChR-library-pirenzepine mixture, one volume is combined with the anti-flag beads (anti-flag/protein A agarose) for IP and the other volume is subjected to a mock IP by combination with the control agarose beads (buffer/protein A agarose beads). Protein A agarose beads are prepared as previously described herein. Similarly, for the ∃-galactosidase mixture, one 20 :L volume is combined with the anti-flag beads for a control IP and the other 20 :L volume is subjected to a mock IP by combination with the control agarose beads. These IPs proceed, mixed by tumbling, for 60 minutes at 4° C. Then each IP is washed with three room temperature cycles of: (1) combining the beads with 1 mL TBS-AG; (2) mixing by tumbling for 2 minutes; and (3) centrifugation at 10,000×g, followed by a careful removal of the supernatant that leaves the pelleted agarose beads in the bottom of the tube. Finally, each 50 :L bed volume bead preparation is then resuspended in an additional 50 :L of TBS (50 mM tris, pH 8.0, 150 mM NaCl) and kept at 4° C. At the completion of this process, four heterogeneous bead preparations are made: (1) m2 mAChR/anti-flag/protein A agarose (m2 beads); (2) m2 mAChR/buffer/protein A agarose beads (m2 control beads); (3) ∃-galactosidase/anti-flag/protein A agarose beads (∃-galactosidase beads), and (4) ∃-galactosidase/buffer/protein A agarose beads ∃-galactosidase control beads).

These bead-based preparations constitute heterogeneous solution phase systems useful for screening in the following manner. Each 100 :L bead preparation is combined with 50 mL of 30% acetonitrile in water heated to 60° C. for 5 minutes to dissociate any bound pirenzepine from the protein. Then the mixture is centrifuged at 10,000×g to pellet the beads, and dissociated protein in the supernatant is transferred to a new tube. The IP procedure allows both an affinity selection of the ligand and the physical separation of the m2 mAChR-ligand complex from unbound library members. These supernatants contain 50 mM tris, pH 8.0, 150 mM NaCl, 80 mM dodecyl ∃-D-maltoside, 10% acetonitrile, and <50 pmol of pirenzepine. Approximately 60 :L of these supernatants are injected into low-flow (100 :L/minute) reverse-phase chromatography (RPC) system. The RPC column (Higgins C-18; 1 mm×50 mm×5 :m) is maintained at 60° C. From this RPC column, the ligand is eluted into a high-resolution mass spectrometer for analysis using a gradient of 5%-95% acetonitrile (0.1% formic acid counterion) in water (w/0.1% formic acid) over 5 minutes.

The mass corresponding to pirenzepine from the supernatant of the m2 mAChR protein control, ∃-galactosidase library test, or ∃-galactosidase control beads should be higher than the pirenzepine mass response in the MS analysis of the m2 mAChR protein beads. It is then inferred that the 2500-member mass-coded library contains a ligand that binds to the m2 mAChR protein with an affinity greater than that of pirenzepine. Libraries that contain hits can thus be detected and selected from among other libraries that do not contain hits.

### EXAMPLE 10

### Enhancing the Hydrophobic Protein Screening Success by Multiplexing

To maintain the amphiphile-solubilized HP in a three-dimensional conformation that enhances the success of screening, HPs are multiplexed in the preparation of screening samples. Multiplexing is defined to mean any method of preparation wherein the target protein is combined with some known molar equivalent of one or more accessory proteins (APs). Five independent criteria are identified to guide the selection of most favorable screening conditions with regard to the use of APs: (1) in the presence of the AP(s), the target HP is observed to bind a known agonist with greater affinity than without the AP(s) present; (2) in the presence of the AP(s), the target HP is observed to bind a known antagonist with greater affinity than without the AP(s) present; (3) in the presence of the AP(s), the HP is shown to have greater functional activity as assayed by enzymatic, in vitro, or cell-based assays; (4) in the presence of the AP(s), the HP is shown to alter its state of multimerization; and (5) in the presence of the AP(s), the HP is shown to have greater conformational stability or uniformity.

As a first example, a method for multiplexed screening of m2 mAChR with G_{∀}_{ᵢ₁}_{∃}_{₁}₍_{₂}-Guanosine-diphosphate (GDP) as a heterotrimeric AP is presented. The G_{∀}_{ᵢ₁}_{∃}_{₁}₍_{₂}-GDP complex was identified as an AP for the m2 mAChR because its presence enhanced the affinity of the m2 mAChR for an antagonist, N-methylscapolamine (NMS) by 5-fold as demonstrated by the following method. ³H-NMS binding assays performed by the method of Rinken and Haga (Rinken, A. and Haga, T. (1993) Arch. Biocehm. Biophys. 301:158-164) showed that the apparent Kd of m2 mAChR for 3H-NMS was 0.17 nM while Kd of the m2 mAChR-G_{∀}_{ᵢ₁}_{∃}_{₁}₍_{₂}-GDP complex was 432 nM. To screen this HP/AP complex, m2 mAChR/G_{∀}_{ᵢ₁}_{∃}_{₁}₍_{₂}-PLs were prepared as described in Example 11 and screened for ligand binding according to any of the methods utilizing affinity selection and ALIS or modified ALIS presented herein with the addition of 50 μM GDP to the binding buffer at the stage of incubation with the ligand compounds.

As a second example, multiplexed screening of heterodimerized 6 and * subtypes of the human opioid receptor is presented. The human 6 opioid receptor was identified as an AP for the human * opioid receptor because its presence caused the multimerization state of the human * opioid receptor to change from monomer to heterodimer as demonstrated by the method of Jordan and Devi (Jordan, B. A. and Devi, L. A., (1999) Nature 399:697-708).

To prepare the */6 opioid receptor heterodimer complex for screening, the following method is used. Human 6-opioid receptor is purified and dialyzed into TBS-AG at a concentration of 50 :M. Human *-opioid receptor is purified and dialyzed into TBS-AG at a concentration of 50 :M. Equal volumes of these two preparations were combined to allow them to dimerize.

The heterodimerized opioid receptor complex is then screened for ligand binding according to any of the methods utilizing affinity selection and ALIS or modified ALIS presented herein.

### EXAMPLE 11

### Methods for the Preparation of Defined GPCR Proteoliposomes (GPCR-PL)

To add conformational stability to detergent-solubilized GPCRs, the detergent micelle solubilization is exchanged for solubilization in defined proteoliposomes meeting the following criteria: (1) the lipid composition of the PL is defined and characterized as having no more than six discrete lipid entities making up 90% of the lipid content of the PL; (2) 90% of the HP-PL preparation has a defined, unimodal size distribution of particles spanning no more than three orders of magnitude and wherein mean particle size is in the range of 5-10000 nm; (c) the PL preparation yields >50 nM HP.

To prepare HP-PL bearing m2 mAChR protein, the following procedure is used. Synthetic lipids (Avanti Polar Lipids, Alabaster, Ala.) D-ribo-phytospingosine-1-phosphate and ceramide-C18:0 each in chloroform are combined in a volume of 1 mL each at 5 mM in a glass test tube, and the chloroform is evaporated at room temperature under a stream of argon for 24 hrs. The lipid residue is then wetted with 0.40 mL of TBS, sonicated for 30 minutes at 28° C., and the mixture is transferred to a 1.5 mL polypropylene tube. To this lipid mixture is added 0.2 mL 50 :M m2 mAChR prepared as described in Peterson, G. L. et al. (Peterson, G. L. et al. (1995) J. Biol. Chem. 270: 17808). This yields a crude mixture of lipid and protein.

The crude protein-lipid mixture is then incubated for 3 hours at 8° C. with 5 minute, 22° C. bath sonication at 30 minute intervals (6 times). This mixture is then subjected to 11 passages through a 100 nm polycarbonate membranes in a small volume extruder according to the manufacturer's protocol (Avanti Polar Lipids, Alabaster, Ala.) at 20° C. This yields a crude PL preparation of 0.6 mL volume, 16 :M m2 mAChR, and excess amphiphile (DbM and lipid). To remove excess lipid and detergent, the crude PL preparation is passed through a 10.0 mL desalting (G-50 sephadex column) previously equilibrated with TBS at 4° C. After application of the crude PL preparation to the desalting column, TBS at 4° C. is used to elute the material from the column. This desalting procedure is conducted with a flow rate of 0.5 mL/minute. The protein concentration is estimated by combining a 10 :L sample with 10 :L of 0.2% Triton X-100 and using that mixture in a Bio-Rad calorimetric protein assay (Bio-Rad Inc., Hercules, Calif.). The concentration is then adjusted by dilution with TBS to 10 :M.

The size distribution is defined using a Coulter N4 submicron particle analyzer following the manufacturer's protocols. An independent assessment of size distribution and mean particle size is accomplished by analytical SEC by injecting 20 :L onto an analytical SEC column with a running buffer TBS at a flow rate of 1.0 mL/minute (G2000_{SWXL}, 5×300 mm, 5 :m; ToSo-Haas), fitted with a UV detector monitoring at 280 nm, and chilled to 12° C. A single peak eluting at 8.23 minutes identified the retention time of the GPCR-PL's which was compared to standard curve of molecular size marker elution times and indicated that the mean apparent size was comparable to that of a 940,000 dalton protein.

Proteoliposomes may also be prepared with HPs complexed with APs. In this procedure, detergent micelle solubilization is exchanged with solubilization in defined proteoliposomes meeting the following criteria: (1) the lipid composition of the PL was defined and characterized as having no more than six discrete lipid entities making up 90% of the lipid content of the PL; (2) 90% of the HP-PL preparation has a defined, unimodal size distribution of particles spanning no more than three orders of magnitude and wherein mean particle size is in the range of 5-10000 nm; (3) the PL preparation yields>50 nM HP; (4) the PL preparation included at least one other protein designated the accessory protein (AP), the presence of which supports the maintenance of a favorable conformation for the target HP.

To prepare HP-PL bearing m2 mAChR, the following procedure was used. Synthetic lipids (Avanti Polar Lipids, Alabaster, Ala.) D-ribo-phytospingosine-1-phosphate and ceramide-C18:0 each in chloroform are combined in a volume of 1 mL each at 5 mM in a glass test tube and the chloroform is evaporated at room temperature under a stream of argon for 24 hrs. The lipid residue is then wetted with 0.40 mL of TBS plus 2 mM MgCl₂ (TBSM), sonicated for 30 minutes at 28° C., and mixture transferred to a 1.5 mL polypropylene tube. Separately, 50 :M G_{∀}_{ᵢ₁}_{∃}_{₁}₍_{₂} was prepared by combining equal volumes of 100 :M G_{∀}_{ᵢ₁} with 100 :M G_{∃}_{₁}₍_{₂} purified as described (ref) and dialyzed into TBSM plus 800 mM DbM (TBSM-AG). To the lipid mixture is added 0.2 mL 50 :M m2 mAChR prepared as described in Peterson, G. L. et al. (Peterson, G. L. et al. (1995) J. Biol. Chem. 270: 17808) and 0.2 mL 50 :M G_{∀}_{ᵢ₁}_{∃}_{₁}₍_{₂}. To this mixture is added 10 :L of 50 mM GDP. This yields a crude mixture of lipid, HP, and AP.

The crude HP/AP-lipid mixture is then incubated for 3 hours at 8° C. with 5 minute, 22° C. bath sonication at 30 minute intervals (6 times). This mixture is then subjected to 11 passages through a 100 nm polycarbonate membranes in a small volume extruder according to the manufacturer's protocol (Avanti Polar Lipids, Alabaster, Ala.) at 20° C. This yields a crude PL preparation of 0.8 mL volume, 12 :M mAChR, 12 :M G_{∀}_{ᵢ₁}_{∃}_{₁}₍_{₂}, and excess amphiphile (DbM and lipid). To remove excess lipid and detergent, the crude PL preparation is passed through a 10.0 mL desalting (G-50 sephadex column) previously equilibrated with TBS at 4° C. After application of the crude PL preparation to the desalting column, TBS at 4° C. is used to elute the material from the column. This desalting procedure is conducted with a flow rate of 0.5 mL/minute. The protein concentration is estimated by combining a 10 :L sample with 10 :L of 0.2% Triton X-100 and using that mixture in a Bio-Rad calorimetric protein assay following the manufacture's instructions. The concentration is then adjusted by dilution with TBS to 20 :M.

To define size distribution, a Coulter N4 submicron particle analyzer is used according to the manufacturers protocol. An independent assessment of size distribution and mean particle size is accomplished by analytical SEC by injecting 20 :L onto an analytical SEC column with a running buffer TBS at a flow rate of 1.0 mL/minute (G2000_{SWXL}, 5×300 mm, 5 :m; ToSo-Haas), fitted with a UV detector monitoring at 280 nm, and chilled to 12° C. A single peak eluting at 8.23 minutes post-injection identified the retention time of the HP/AP-PL's which is compared to standard curve of molecular size marker elution times. The resultant mean apparent size is comparable to that of a 940,000 dalton protein.

Control AP-PLs for use as screening controls are made to meet the following criteria: (1) the lipid composition of the PL was defined and characterized as having no more than six discrete lipid entities making up 90% of the lipid content of the PL; (2) 90% of the HP-PL preparation has a defined, unimodal size distribution of particles spanning no more than three orders of magnitude and wherein mean particle size is in the range of 5-10000 nm; (3) the PL preparation is designed to exactly mimic the analogous HP/AP-PL except no target HP is present.

To prepare AP-PL bearing m2 mAChR, the following procedure is used. Synthetic lipids (Avanti Polar Lipids, Alabaster, Ala.) D-ribo-phytospingosine-1-phosphate and ceramide-C18:0 each in chloroform are combined in a volume of 1 mL each at 5 mM in a glass test tube and the chloroform is evaporated at room temperature under a stream of argon for 24 hours. The lipid residue is then wetted with 0.40 mL of TBS plus 2 mM MgCl₂ (TBSM), sonicated for 30 minutes at 28° C., and mixture transferred to a 1.5 mL polypropylene tube. Separately, 50 :M G_{∀}_{ᵢ₁}_{∃}_{₁}₍_{₂} was prepared by combining equal volumes of 100 :M G_{∀}_{ᵢ₁} with 100 :M G_{∃}_{₁}₍_{₂} purified as described Hou, Y. et al., J. Biol. Chem. (2000) 275:38961-6 and dialyzed into TBSM plus 800 mM DbM (TBSM-AG). To the lipid mixture is added 0.2 mL TBS-AG and 0.2 mL 50 :M G_{∀}_{ᵢ₁}_{∃}_{₁}₍_{₂}. To this mixture is added 10 :L of 50 mM GDP. This yields a crude mixture of lipid and AP. The protein concentration is estimated by combining a 10 :L sample with 10 :L of 0.2% Triton X-100 and using that mixture in a Bio-Rad calorimetric protein assay following the instructions of the manufacturer. The concentration is then adjusted by dilution with TBS to 10 :M.

This crude AP-lipid mixture is then incubated for 3 hours at 8° C. with 5 minute, 22° C. bath sonication at 30 minute intervals (6 times). This mixture is then subjected to 11 passages through a 100 nm polycarbonate membranes in a small volume extruder according to the manufacturer's protocol (Avanti Polar Lipids, Alabaster, Ala.) at 20° C. This yields a crude PL preparation of 0.8 mL volume, 12 :M mAChR, 12 :M G_{∀}_{ᵢ₁}_{∃}_{₁}₍_{₂}, and excess amphiphile (DbM and lipid). To remove excess lipid and detergent, the crude PL preparation is passed through a 10.0 mL desalting (G-50 sephadex column) previously equilibrated with TBS at 4° C. After application of the crude PL preparation to the desalting column, TBS at 4° C. is used to elute the material from the column. This desalting procedure is conducted with a flow rate of 0.5 mL/minute.

To define size distribution, a Coulter N4 submicron particle analyzer is used according to the manufacturers protocol. An independent assessment of size distribution and mean particle size is accomplished by analytical SEC by injecting 20 :L onto an analytical SEC column with a running buffer TBS at a flow rate of 1.0 mL/minute (G2000_{SWXL}, 5×300 mm, 5 :m; ToSo-Haas), fitted with a UV detector monitoring at 280 nm, and chilled to 12° C. A single peak eluting at 8.23 minutes identified the retention time of the AP-PL's which is compared to standard curve of molecular size marker elution times. The resultant mean apparent size is comparable to that of a 940,000 dalton protein.

The proteoliposomes described herein are then screened for ligand binding according to any of the methods utilizing affinity selection and ALIS or modified ALIS presented herein.

### EXAMPLE 12

### Dual Epitope Affinity Purification of HPs

The construction of nucleic acid sequences encoding the tagged HP proteins of the invention is well within the skill of those in the art, utilizing routing procedures common in the art of nucleic acid cloning.

Tagged-HPs, e.g., Mellitin-Flag Tag-Human m1 mAChR-EE-tag protein or Mellitin-Flag Tag-Human Beta 2 Adrenergic Receptor-EE-tag protein, are produced from a recombinant baculovirus following the methodology provided by the manufacturer of the viral expression system (Pharmingen, San Diego, Calif.).

Briefly, recombinant virus is selected based on its ability to direct the expression of the recombinant protein(s). Protein expression is confirmed by western blot analysis of detergent-solubilized cell lysates of the infected insect cells. Using primary antibodies that recognize either the Flag epitope, the EE epitope, or the HP protein itself, the western blot reveals whether or not, and to what degree, the HP proteins are expressed. If possible, a functional assay is performed to determine that the expressed protein is functional. For example, for the Mellitin-Flag Tag-Human m1 mAChR-EE-tag protein, a cell-based ³H-N-methylscapolamine binding analyses is performed by the method of Rinken and Haga (Rinken, A. and Haga, T. (1993) Arch. Biocehm. Biophys. 301:158-164)confirmed that the virus-directed protein expression was functional, indicating a Bmax of 0.5×10⁶ receptors per cell and a Kd of 0.10 nM. This baculovirus was then amplified by to a high titer of 2.0×10⁸ pfu/mL by conventional methods (Pharmingen, San Diego, Calif.).

Next, large-scale insect cell cultures are obtained for the isolation of the proteins. Briefly, once high titer virus stocks are generated the constructs of interest, ten 10 liter production runs were executed, growing Sf21 insect cells to a density of 2×10⁶ cells/mL in a bioreactor with wave agitation (Wave Biotechnology, Bedminster, N.J.). For each protein produced, these cells were inoculated with high titer virus stock at an multiplicity of infection (MOI) of 5 and two days post-infection the cells were harvested.

The HP proteins are then solubilzed. For example, mAChR protein expressing cells are collected by centrifugation for 30 minutes at 10,000×g. The cell pellets from all ten bioreactor production runs are combined, and the cell pellets are suspended in 500 mL of ice cold TBS buffer plus 1 mM EDTA, 10 :g/ml pepstatin and 1 :g/ml pmethylsulfonylfluoride, and 20 mM dodecyl-∃-D-maltoside (DbM). This cell slurry is subjected to 50 strokes in a pestle A dounce homogenizer at 4° C. to break open the cells and solubilize the membrane proteins. To clarify this suspension, the material is centrifuged for 60 minutes at 40000×g at 4° C. to remove insoluble material. The supernatant is then collected and used as a source of soluble Mellitin-Flag Tag-Human m1 mAChR-EE protein. (Note: The honey bee mellitin signal sequence is cleaved off by cellular proteases in the process of expression and trafficking to the plasma membrane of the insect cell. From this point on the solubilized protein is referred to as Flag Tag-Human m1 mAChR-EE.)

Each solubilized recombinant HP is then purified with two rounds of affinity purification over an antibody affinity column. Both the anti-EE-tag and anti-Flag-tag columns are prepared similarly. Briefly, 35 mg of purified anti-epitope antibody is dialyzed into coupling buffer and coupled to 10 mL CNBr-activated sepharose beads according the manufacturers protocol (Amersham Pharmacia, Piscataway, N.J.). The resin is then transferred to a 1.2×15 cm polypropylene column, and the epitope affinity columns are then equilibrated with TBS-AG at 4° C.

Next, 50 mL of solubilized HP protein, e.g., FlagTag-Human m1 mAChR-EE, is applied to the anti-EE affinity column at a flow rate of 0.2 mL per minute. The column was then washed with 5 column volumes of TBS-AG at the same flow rate. The specifically-bound HP is then eluted from the column with a bolus of excess EE peptide (NH2-EEEEYMPME-COOH; Sigma-Genosys, St. Louis, Mo.) solubilized in a volume of 15 mL in TBS-AG at 10 mM. The eluant is then applied similarly to the anti-Flag-tag affinity column, similarly washed, and eluted from the column with excess anti-FLAG peptide (NH2-DYKDDDDK-COOH; Sigma-Genosys, St. Louis, Mo.). This material is then dialyzed with two exchanges into a 100 fold volume of TBS-AG overnight at 4° C.

Sucrose gradient ultracentrifugation is then used to remove misfolded polypeptide. Briefly, the material is applied to a discontinuous step gradient of 5%-25% sucrose in TBS-AG and centrifuged in a Beckman SW Ti.50 rotor at 100000×g for 4 hours at 4° C. The properly conformed HP, e.g., FlagTag-Human m1 mAChR-EE, material is collected at the 5%-25% interface. This material is then subjected to a final dialysis with two exchanges into a 100 fold volume of TBS-AG overnight at 4° C.

The protein concentration of the HP solution is determined by colorimetric protein assay (Bio-Rad Laboratories, Inc., Hercules, Calif.) and adjusted as necessary, by dilution or concentration in a stirred ultrafiltration cell (Millipore, Bedford, Mass.), to 50 :M. The detergent concentration is also determined by RPC-UV using a Higgins C-18 column whereupon 50 :L of the mixture the ligand is eluted into a high-resolution mass spectrometer for analysis using a gradient of 5%-95% acetonitrile (0.1% formic acid counterion) in water (w/0.1% formic acid) over 20 minutes. The Rt of the DbM is determined by a separate control experiments under identical conditions. To quantitate the amount of detergent, the area under the DbM peak from the protein sample is compared to a standard curve generated from multiple RPC runs with identical conditions assaying the DbM peak heigth for DbM samples of known concentration. If the detergent concentration is estimated to be below 0.48 mM (4×cmc), it is adjusted with the addition of a small amount of concentrated DbM in TBS.

### EXAMPLE 13

### Epitope Affinity and Metal Chelate Affinity Chromatography (MCAC) of HPs

HPs are also constructed with an epitope affinity tag and a metal chelate affinity tag, as represented, for example, by Hemagglutinin SS-Rat m3 mAChR-HSV-OctaHis. Briefly, an isolated nucleic acid molecule encoding Hemagglutinin SS-Rat m3 mAChR-HSV-OctaHis is used in the production of a recombinant baculovirus following methods provided by the manufacturer (Pharmingen, San Diego, Calif.). Virus production, the large-scale insect cell culture and expression of protein, the preparation of epitope affinity columns, the preparation of solubilized HP, and epitope affinity purification were all performed as previosly described herein except that anti-HSV antibody replaces the anti-FLAG antibody in the preparation of the HSV epitope affinity column and the HSV peptide (NH2-QPELAPEDPED-COOH; Sigma-Genosys) is used to elute the Hemagglutinin SS-Rat m3 mAChR-HSV-OctaHis protein from the column instead of the FLAG peptide. Also, since there is no EE epitope on the Hemagglutinin SS-Rat m3 mAChR-HSV-OctaHis protein, no epitope affinity purification using the EE epitope is used. (Note: The Hemagglutinin SS signal sequence may be cleaved off in some cell lines by cellular proteases in the process of expression and trafficking to the plasma membrane of the insect cell. From this point on the solubilized protein is referred to as Rat m3 mAChR-HSV-OctaHis.

For the final affinity purification step, Rat m3 mAChR-HSV-OctaHis is applied, at a flow rate of 0.2 mL/minutes, to a MCAC column prepared by loading 10 mL of Ni-NTA resin (Qiagen) into a 1.2×15 cm polypropylene column and equilibrating the column with 100 mL of TBS-AG at 4° C. at a flow rate of 0.2 mL/minutes. Once the Rat m3 mAChR-HSV-OctaHis is bound to the column, the column is washed with 100 mL of TBS-AG containing 5 mM imidazole. Then, the column is developed with a 50 mL linear gradient of 5 mM-350 mM imidazole in TBS-AG at the same flow rate, collecting 0.5 mL fractions. Fractions containing active mAChR, as assessed by a known radioligand binding assay, elute at imidazole concentrations of 190-240 mM in a volume of 11 mL. This material is then dialyzed with three exchanges into a 100 fold volume of TBS-AG overnight at 4° C. to remove excess imidazole. The material is then subjected to sucrose gradient ultracentrifugation and the concentrations of protein and detergent are estimated and adjusted as previously described herein.

### EXAMPLE 14

### Characterization of Purified HPs

To confirm the purity of the final preparation of the HP(s) produced herein, each sample is subjected to SDS-PAGE analysis on 5-12% Novex (Invitrogen, San Diego, Calif.) gels according to the manufacturer's instructions. Ten :g of sample are loaded per gel lane, and the protein samples are visualized by silver staining (Peterson, G. L. et al. (1995) J. Biol. Chem. 270: 17808). The specific activities of the mAChR proteins prepared according to these methods are determined according the method of Peterson, G. L. et al. (Peterson, G. L. et al. (1995) J. Biol. Chem. 270: 17808). For the methods described herein, typically the specific activity of the proteins is determined to be in the range of 11-16 nmol of specific ligand binding per mg mAChR protein.

Ligand affinity chromatography is another measure by which the HPs of the invention may be evaluated for specific activity. For example, the mAChR purified by the methods described herein may be subjected to known ligand affinity chromatography over a column of immobilized mAChR ligand 3-(2′-aminobenzhydryloxy)-tropane(ABT).

Equivalents

Those skilled in the art will recognize, or be able to ascertain, using no more than routine experimentation, many equivalents to the specific embodiments of the invention described herein. Such equivalents are intended to be encompassed by the following claims.

What is claimed is:

1. A method for identifying a ligand for a hydrophobic protein, the method comprising
(a) selecting a ligand molecule by affinity selection by exposing a hydrophobic target protein bound by an amphiphile to a multiplicity of molecules to promote the formation of at least one complex between the hydrophobic target protein and the ligand molecule,
(b) separating the complex from the unbound molecules, and
(c) identifying the ligand molecule.

2. The method of claim 1, wherein exposure of the hydrophobic target protein to a multiplicity of molecules occurs under homogeneous solution phase conditions.

3. The method of claim 1, wherein exposure of the hydrophobic target protein to a multiplicity of molecules occurs under heterogeneous solution phase conditions.

4. The method of claim 1, wherein selection of the ligand molecule is done using multi-dimensional chromatography.

5. The method of claim 1, wherein the hydrophobic target protein is selected from the group consisting of:
(a) of a membrane protein,
(b) an integral membrane protein,
(c) a transmembrane protein,
(d) a monotopic membrane protein,
(e) a polytopic membrane protein,
(f) a pump protein,
(g) a channel protein,
(h) a receptor kinase protein,
(i) a G protein-coupled receptor protein,
(j) a membrane-associated enzyme, and
(k) a transporter protein.

6. The method of claim 1, wherein the multiplicity of molecules is a mass-coded library of molecules.

7. The method of claim 1, wherein the multiplicity of molecules is a library of molecules that is not mass-coded.

8. The method of claim 1, wherein the amphiphile is selected from the group consisting of:
(a) a polar lipid,
(b) an amphiphilic macromolecular polymer,
(c) a surfactant or detergent, and
(d) an amphiphilic polypeptide.

9. The method of claim 1, wherein ligand molecule identification is done by mass spectral analysis.

10. The method of claim 1, wherein the ligand molecule is deconvoluted by mass spectral analysis.

11. The method of claim 1, wherein separation of the complex from the unbound molecules is accomplished with solid phase chromatography media.

12. The method according to claim 1, wherein the hydrophobic target protein comprises
(a) at least one transmembrane domain sequence,
(b) at least two tag sequences useful for affinity selection, and
(c) a hydrophobic protein (HP) sequence.

13. The method according to claim 12, wherein the hydrophobic protein sequence is selected from the group consisting of
(a) of a membrane protein,
(b) an integral membrane protein,
(c) a transmembrane protein,
(d) a monotopic membrane protein,
(e) a polytopic membrane protein,
(f) a pump protein,
(g) a channel protein,
(h) a receptor kinase protein,
(i) a G protein-coupled receptor protein,
(j) a membrane-associated enzyme, and
(k) a transporter protein.

14. The method according to claim 12, wherein the tag sequences comprise epitope tag sequences selected from the group consisting of
(a) a FLAG tag (NH2-DYKDDDDK-COOH) (SEQ ID NO:29),
(b) an EE tag (NH2-EEEEYMPME-COOH) (SEQ ID NO:30),
(c) a hemagglutinin tag (NH2-YPYDVPDYA-COOH) (SEQ ID NO:31),
(d) a myc tag (NH2-KHKLEQLRNSGA-COOH) (SEQ ID NO:32), and
(e) an HSV tag (NH2-QPELAPEDPED-COOH) (SEQ ID NO:33).

15. The method according to claim 12, wherein the hydrophobic target protein comprises a sequence with an amino terminus to carboxy terminus order selected from the group consisting of
(a) Tag1-Tag2-HP,
(b) Tag1-HP-Tag2, and
(c) HP-Tag1-Tag2.

16. The method according to claim 15, wherein the hydrophobic target protein is selected from the group consisting of
(a) Myc tag-EE tag-Human m2 mAChR (SEQ ID NO:7),
(b) Flag tag-Human Beta 2 Adrenergic Receptor-EE tag (SEQ ID NO:8),
(c) Human Neurokinin 3 Receptor-HSV tag-Myc tag (SEQ ID NO:9),
(d) Flag tag-Human m1 mAChR-EE tag (SEQ ID NO:10), and
(e) Rat m3 mAChR-HSV tag-OctaHis tag (SEQ ID NO:11).

17. The method according to claim 15, wherein the hydrophobic target protein further comprises a heterologous signal sequence (SS) at the amino terminus.

18. The method according to claim 17, wherein the heterologous signal sequence is selected from the group consisting of
(a) the Mellitin signal sequence of NH₂-KFLVNVALVFMVVYISYIYA-COOH (SEQ ID NO:12),
(b) the GP signal sequence of NH₂-VRTAVLILLLVRFSEP-COOH COOH (SEQ ID NO:13),
(c) the Hemagglutinin signal sequence of NH₂-KTIIALSYIFCLVFA-COOH (SEQ ID NO:14),
(d) the rhodopsin tag 1 signal sequence of NH₂-MNGTEGPNFYVPFSNKTGVVRSPFEAPQYYLAEP-COOH (SEQ ID NO:15), and
(e) the rhodopsin tag ID4 signal sequence of NH₂-GKNPLGVRKTETSQVAPA-COOH (SEQ ID NO:16).

19. The method according to claim 18, wherein the tag sequences further comprise a hexahistidine sequence (SEQ ID NO:17) and a decahistidine sequence (SEQ ID NO:18).

20. The method according to claim 19, wherein the hydrophobic target protein is selected from the group consisting of
(a) GP67 SS-Myc tag-EE tag-Human m2 mAChR (SEQ ID NO:19),
(b) Mellitin SS-Flag tag-Human Beta 2 Adrenergic Receptor-EE tag(SEQ ID NO:20),
(c) Hemagglutinin SS-Human Neurokinin 3 Receptor-HSV tag-Myc tag (SEQ ID NO:21),
(d) Mellitin SS-Flag tag-Human m1 mAChR-EE tag (SEQ ID NO:22), and
(e) Hemagglutinin SS-Rat m3 mAChR-HSV tag-OctaHis tag (SEQ ID NO:23).

21. A method of isolating a hydrophobic protein, the method comprising
(a) purifying the hydrophobic protein by sucrose gradient ultracentrifugation,
(b) purifying the hydrophobic protein by antibody affinity purification, and
(c) purifying the hydrophobic protein by immobilized metal affinity chromatography.

22. The method of claim 21, wherein the hydrophobic protein comprises
(a) at least one transmembrane domain sequence,
(b) at least two tag sequences useful for affinity selection, and
(c) a hydrophobic protein (HP) sequence.

23. The method according to claim 22, wherein the hydrophobic protein sequence is selected from the group consisting of
(a) a membrane protein,
(b) an integral membrane protein,
(c) a transmembrane protein,
(d) a monotopic membrane protein,
(e) a polytopic membrane protein,
(f) a pump protein,
(g) a channel protein,
(h) a receptor kinase protein,
(i) a G protein-coupled receptor protein,
(j) a membrane-associated enzyme, and
(k) a transporter protein.

24. The method according to claim 22, wherein the tag sequences comprise epitope tag sequences selected from the group consisting of
(a) a FLAG tag (NH2-DYKDDDDK-COOH) (SEQ ID NO:29),
(b) an EE tag (NH2-EEEEYMPME-COOH) (SEQ ID NO:30),
(c) a hemagglutinin tag (NH2-YPYDVPDYA-COOH) (SEQ ID NO:31),
(d) a myc tag (NH2-KHKLEQLRNSGA-COOH) (SEQ ID NO:32), and
(e) an HSV tag (NH2-QPELAPEDPED-COOH) (SEQ ID NO:33).

25. The method according to claim 22, wherein the hydrophobic protein comprises a sequence with an amino terminus to carboxy terminus order selected from the group consisting of
(a) Tag1-Tag2-HP,
(b) Tag1-HP-Tag2, and
(c) HP-Tag1-Tag2.

26. The method according to claim 22, wherein the hydrophobic protein is selected from the group consisting of
(a) Myc tag-EE tag-Human m2 mAChR (SEQ ID NO:7),
(b) Flag tag-Human Beta 2 Adrenergic Receptor-EE tag (SEQ ID NO:8),
(c) Human Neurokinin 3 Receptor-HSV tag-Myc tag (SEQ ID NO:9),
(d) Flag tag-Human m1 mAChR-EE tag (SEQ ID NO:10), and
(e) Rat m3 mAChR-HSV tag-OctaHis tag (SEQ ID NO:11).

27. The method according to claim 22, wherein the hydrophobic protein further comprises a heterologous signal sequence (SS) at the amino terminus.

28. The method according to claim 27, wherein the heterologous signal sequence is selected from the group consisting of
(a) the Mellitin signal sequence of NH₂-KFLVNVALVFMVVYISYIYA-COOH (SEQ ID NO:12),
(b) the GP signal sequence of NH₂-VRTAVLILLLVRFSEP-COOH (SEQ ID NO:13),
(c) the Hemagglutinin signal sequence of NH₂-KTIIALSYIFCLVFA-COOH (SEQ ID NO:14),
(d) the rhodopsin tag 1 signal sequence of NH₂-MNGTEGPNFYVPFSNKTGVVRSPFEAPQYYLAEP-COOH (SEQ ID NO:15), and
(e) the rhodopsin tag ID4 signal sequence of NH2-GKNPLGVRKTETSQVAPA-COOH (SEQ ID NO:16).

29. The method according to claim 14, wherein the tag sequences further comprise a hexahistidine sequence (SEQ ID NO:17) and a decahistidine sequence (SEQ ID NO:18).

30. The method according to claim 29, wherein the hydrophobic target protein is selected from the group consisting of
(a) GP67 SS-Myc tag-EE tag-Human m2 mAChR (SEQ ID NO:19),
(b) Mellitin SS-Flag tag-Human Beta 2 Adrenergic Receptor-EE tag(SEQ ID NO:20),
(c) Hemagglutinin SS-Human Neurokinin 3 Receptor-HSV tag-Myc tag (SEQ ID NO:21),
(d) Mellitin SS-Flag tag-Human m1 mAChR-EE tag (SEQ ID NO:22), and
(e) Hemagglutinin SS-Rat m3 mAChR-HSV tag-OctaHis tag (SEQ ID NO:23).

31. An isolated nucleic acid molecule suitable for hydrophobic protein expression, comprising
(a) a vector polynucleotide sequence for protein expression in a eukaryotic cell, and
(b) a polynucleotide sequence encoding an engineered hydrophobic protein comprising the following elements
(i) an N-terminal methionine residue,
(ii) a heterologous signal sequence (SS),
(iii) at least one transmembrane domain sequence,
(iv) at least two tag sequences useful for affinity selection, and
(v) a hydrophobic protein (HP) sequence.

32. The isolated nucleic acid molecule of claim 32, wherein the N-terminal methionine sequence and the heterologous signal sequence are selected from the group consisting of
(a) MKFLVNVALVFMVVYISYIYA (SEQ ID NO:24),
(b) MVRTAVLILLLVRFSEP (SEQ ID NO:25),
(c) MKTIIALSYIFCLVFA (SEQ ID NO:26)
(d) MMNGTEGPNFYVPFSNKTGVVRSPFEAPQYYLAEP-COOH (SEQ ID NO:27) and
(e) MGKNPLGVRKTETSQVAPA-COOH (SEQ ID NO:28).

33. The isolated nucleic acid molecule of claim 33, wherein the tag sequences comprise epitope tag sequences selected from the group consisting of
(a) a FLAG tag (NH2-DYKDDDDK-COOH) (SEQ ID NO:1),
(b) an EE tag (NH2-EEEEYMPME-COOH) (SEQ ID NO:2),
(c) a hemagglutinin tag (NH2-YPYDVPDYA-COOH) (SEQ ID NO:3),
(d) a myc tag (NH2-KHKLEQLRNSGA-COOH) (SEQ ID NO:4), and
(e) an HSV tag (NH2-QPELAPEDPED-COOH) (SEQ ID NO:5).

34. The isolated nucleic acid molecule of claim 33, wherein the elements of the engineered hydrophobic protein are arrayed from an amino to carboxy terminus order selected from the group consisting of
(a) SS-Tag1-Tag2-HP,
(b) SS-Tag1-HP-Tag2, and
(c) SS-HP-Tag1-Tag2.

35. The isolated nucleic acid molecule of claim 34, wherein the tag sequences further comprise a hexahistidine sequence (SEQ ID NO:17) and a decahistidine sequence (SEQ ID NO:18).

36. The isolated nucleic acid molecule of claim 35, wherein the engineered hydrophobic protein is selected from the group consisting of
(a) GP67-Myc-EE-Human m2 mAChR (SEQ ID NO:19),
(b) Mellitin-Flag Tag-Human m1 mAChR-EE (SEQ ID NO:20), and

37. A method for identifying a ligand for a hydrophobic protein, the method comprising
(a) selecting a hydrophobic target protein from the group consisting of
(i) of a membrane protein,
(ii) an integral membrane protein,
(iii) a transmembrane protein,
(iv) a monotopic membrane protein,
(v) a polytopic membrane protein,
(vi) a pump protein,
(vii) a channel protein,
(viii) a receptor kinase protein,
(ix) a G protein-coupled receptor protein,
(x) a membrane-associated enzyme, and
(xi) a transporter protein,
(xii) wherein the hydrophobic protein is bound by amphiphile;
(b) selecting an amphiphile to bind the hydrophobic protein from the group consisting of:
(i) a polar lipid,
(ii) an amphiphilic macromolecular polymer,
(iii) a surfactant or detergent, and
(iv) an amphiphilic polypeptide;
(c) selecting a ligand molecule using multi-dimensional chromatography by affinity selection by exposing under homogenous solution phase conditions the hydrophobic target protein bound by an amphiphile to a multiplicity of molecules from a mass-coded library to promote the formation of at least one complex between the hydrophobic target protein and the ligand molecule;
(d) separating the complex from the unbound molecules; and
(e) identifying the ligand molecule by mass spectral analysis.

38. A method for identifying a ligand for a hydrophobic protein, the method comprising
(a) selecting a hydrophobic target protein from the group consisting of
(i) of a membrane protein,
(ii) an integral membrane protein,
(iii) a transmembrane protein,
(iv) a monotopic membrane protein,
(v) a polytopic membrane protein,
(vi) a pump protein,
(vii) a channel protein,
(viii) a receptor kinase protein,
(ix) a G protein-coupled receptor protein,
(x) a membrane-associated enzyme, and
(xi) a transporter protein,
(xii) wherein the hydrophobic protein is bound by amphiphile;
(b) selecting an amphiphile to bind the hydrophobic protein from the group consisting of:
(i) a polar lipid,
(ii) an amphiphilic macromolecular polymer,
(iii) a surfactant or detergent, and
(iv) an amphiphilic polypeptide;
(c) selecting a ligand molecule using multi-dimensional chromatography by affinity selection by exposing under heterogeneous solution phase conditions a hydrophobic target protein bound by an amphiphile to a multiplicity of molecules from a library that is not mass-coded to promote the formation of at least one complex between the hydrophobic target protein and the ligand molecule;
(d) separating the complex from the unbound molecules,;and
(e) identifying the ligand molecule by mass spectral analysis.

39. A method of isolating a hydrophobic protein, the method comprising
(a) selecting a hydrophobic protein comprising
(i) at least one transmembrane domain sequence,
(ii) at least two tag sequences useful for affinity selection,selected from the group consisting of:
(1) a FLAG tag (NH2-DYKDDDDK-COOH) (SEQ ID NO:29),
(2) an EE tag (NH2-EEEEYMPME-COOH) (SEQ ID NO:30),
(3) a hemagglutinin tag (NH2-YPYDVPDYA-COOH) (SEQ ID NO:31),
(4) a myc tag (NH2-KHKLEQLRNSGA-COOH) (SEQ ID NO:32), and
(5) an HSV tag (NH2-QPELAPEDPED-COOH) (SEQ ID NO:33);
(iii) a hydrophobic protein (HP) sequence selected from the group consisting of:
(1) a membrane protein,
(2) an integral membrane protein,
(3) a transmembrane protein,
(4) a monotopic membrane protein,
(5) a polytopic membrane protein,
(6) a pump protein,
(7) a channel protein,
(8) a receptor kinase protein,
(9) a G protein-coupled receptor protein,
(10) a membrane-associated enzyme, and
(11) a transporter protein;
(b) purifying the hydrophobic protein by sucrose gradient ultracentrifugation;
(c) purifying the hydrophobic protein by antibody affinity purification;and
(d) purifying the hydrophobic protein by immobilized metal affinity chromatography.

40. An isolated nucleic acid molecule suitable for hydrophobic protein expression, comprising
(a) a vector polynucleotide sequence for protein expression in a eukaryotic cell; and
(b) a polynucleotide sequence encoding an engineered hydrophobic protein comprising the following elements
(i) an N-terminal methionine residue,
(ii) a heterologous signal sequence (SS), wherein the N-terminal methionine sequence and the heterologous signal sequence are selected from the group consisting of
(1) MKFLVNVALVFMVVYISYIYA (SEQ ID NO:24),
(2) MVRTAVLILLLVRFSEP (SEQ ID NO:25),
(3) MKTIIALSYIFCLVFA (SEQ ID NO:26)
(4) MMNGTEGPNFYVPFSNKTGVVRSPFEAPQYYL AEP-COOH (SEQ ID NO:27) and
(5) MGKNPLGVRKTETSQVAPA-COOH (SEQ ID NO:28),
(iii) at least one transmembrane domain sequence;
(iv) at least two tag sequences useful for affinity selection selected from the group consisting of
(1) a FLAG tag (NH2-DYKDDDDK-COOH) (SEQ ID NO:1),
(2) an EE tag (NH2-EEEEYMPME-COOH) (SEQ ID NO:2),
(3) a hemagglutinin tag (NH2-YPYDVPDYA-COOH) (SEQ ID NO:3),
(4) a myc tag (NH2-KHKLEQLRNSGA-COOH) (SEQ ID NO:4), and
(5) an HSV tag (NH2-QPELAPEDPED-COOH) (SEQ ID NO:5), and
(v) a hydrophobic protein (HP) sequence selected from the group consisting of:
(1) a membrane protein,
(2) an integral membrane protein,
(3) a transmembrane protein,
(4) a monotopic membrane protein,
(5) a polytopic membrane protein,
(6) a pump protein,
(7) a channel protein,
(8) a receptor kinase protein,
(9) a G protein-coupled receptor protein,
(10) a membrane-associated enzyme, and
(11) a transporter protein.
